SEQUENCE LISTING (34 sequences)

SEQ ID NO: 1 - protein, 453 aa, Solanum lycopersicon
MGTQVTEHGTSNLRVVMFPWLAYGHISPFLYVAKKLADRGFLIYLCSTPINLKSTIEKIPEKYADSIHLIELHLPELPELPPHYHTTNGLPPHLNHTLQKALKMSKPNLSKILKNLKPDLMIYDVLQQWAERVANEQSIPAVRLLTFGAAVFSYFCNLVKKPGVEFPFPDIYLRKIEQVKLGEMLEKSAKDQDPDDEERLVDEYKQIALICTSRTIEAKYIDFLLELSNLKVVPVGSPVQDLITNDADDMELIDWLGSKDENSTVFVSFGSEYFLSKEDMEEVALGLELSNVNFVWVARFPKGEEQNLEDALPKGFLERIGERGRVLDKFAPQLRILNHTSTGGFISHCGWNSVMESIHFGVPIVAMPMHLDQPMNARLIVELGVAVEIVRDDDGKIYREEIAKTLKDVITERIGENLRAKMREISKNLNSISGEEMDAAAHELIQFCKISTN

SEQ ID NO: 2 - protein, 444 aa, Solanum lycopersicon
MEAKKNKMSILMLPWLAHGHISPFLELAKKLTNRNFHIYMCSTPINLSSIKKNITKKYFESIELVEFHLPSLPNLPPHYHTTNGLPPHLMNTLKTAFENASPNFSKILQTLNPDLVIYDFNQPWAAESASSVNIPAVQFLTFGAAVVSLAIHMFEDTEDKFPFPEIYLHEYEMLSLKEAVKEAPGNKYSFDEAIRLSRDIVLVKTCRDFEGKYVDYLSNLVSKKIVPVGSLVQESIARDDNDEEIMQWLDKKEKGLTVFVSFGSEYFLSKEDIFVVARGLELSKVNFIWVIRFSQGERISIQDALPEGYLERVGERGMVIEGWAPQAMILQHPSIGGFVSHCGWSSFMESMKFGVPIIAMPMHIDQPMNARLVEYIRMGVEAARDENGKLQSEEIANTIRKVLVEESGEDVRNKAKELSGKMNAKGDEEIDGVVEELMALCSNK

SEQ ID NO: 3 - protein, 454 aa, Solanum lycopersicon
MDGSNEKSIRVLMFPWLGHGHISPFFELAKKLVKRNFTIFLVSTPANFISIKQKLIHENLCDKIHLFDLRLPSLPDLPPHYHTTNGLPPHLMSTLKKAFAKSRPIFTQIMNTIEPDLLLYDLLQPWAPKVAKEKNIPSVVFVTSSATMFSYMFHNFRYPNSQFPFSSIYYRDYELTRLIKNQEMETIEQHQRDNKSVKMCFKRSTNIVLIKGFKEIDGQYCEYISSLTKKRVVPVGPLVQEQTSEDNNSQILTWLNQKSKGSTIFVSFGSEYFLSQEDREEIAHGLEQSRVNFIWVVRFPKGEKLKLEQALPRDFFKKVGERGMVVEDWAPQAKILGNPNIGGFVSHCGWNSVLESMKIGVPIIAMPMHLDQPLNARLVEEVGIGLEVVRDKDGKLDGEQISEIINKVVLEKEGESIREKAKKMSETIRVKGDEEIDDVVQELVNLCKTRSNVV

SEQ ID NO: 4 - protein, 446 aa, Solanum lycopersicon
MSTTLKVLMFPFLAYGHISPYLNVAKKLADRGFLIYLCSTPINLKSTINKIPEKYADSIQLIELHLPELPELPPHYHTTNGLPPNLNHILRRALKMSKPNFSKIMQNLKPDLLIYDILQQWAEDVATELNIPAVKLLTSGVAVFSYFFNLTKKPEVEFPYPAIYLRKIELVRWCETLSKHNKEGEEHDDGLAYGNMQIMLMSTSKILEAKYIDYCIELTNWKVVPVGSLVQDSITNDAADDDMELIDWLGTKDENSTVFVSFGSEYFLSKEDVEEVAFGLELSNVNFIWVVRFPKGEEKNLEDVLPKGFFERIGERGRVLDKFAPQPRILNHPSTGGFISHCGWNSAMESIDFGVPIVAMPMQLDQPMNARLIVELGVAVEIVRDDDGKIYRGEIAETLKGVITGEIGEILRAKVRDISKNLKAIKDEEMDVAAQELIQLCRNSNK

SEQ ID NO: 5 - protein, 473 aa, Stevia rebaudiana
MATSDSIVDDRKQLHVATFPWLAFGHILPYLQLSKLIAEKGHKVSFLSTTRNIQRLSSHISPLINVVQLTLPRVQELPEDAEATTDVHPEDIPYLKKASDGLQPEVTRFLEQHSPDWIIYDYTHYWLPSIAASLGISRAHFSVTTPWAIAYMGPSADAMINGSDGRTTVEDLTTPPKWFPFPTKVCWRKHDLARLVPYKAPGISDGYRMGLVLKGSDCLLSKCYHEFGTQWLPLLETLHQVPVVPVGLLPPEIPGDEKDETWVSIKKWLDGKQKGSVVYVALGSEVLVSQTEVVELALGLELSGLPFVWAYRKPKGPAKSDSVELPDGFVERTRDRGLVWTSWAPQLRILSHESVCGFLTHCGSGSIVEGLMFGHPLIMLPIFGDQPLNARLLEDKQVGIEIPRNEEDGCLTKESVARSLRSVVVEKEGEIYKANARELSKIYNDTKVEKEYVSQFVDYLEKNARAVAIDHES

SEQ ID NO: 6 - protein, 481 aa, Stevia rebaudiana
MDAMATTEKKPHVIFIPFPAQSHIKAMLKLAQLLHHKGLQITFVNTDFIHNQFLESSGPHCLDGAPGFRFETIPDGVSHSPEASIPIRESLLRSIETNFLDRFIDLVTKLPDPPTCIISDGFLSVFTIDAAKKLGIPVMMYWTLAACGFMGFYHIHSLIEKGFAPLKDASYLTNGYLDTVIDWVPGMEGIRLKDFPLDWSTDLNDKVLMFTTEAPQRSHKVSHHIFHTFDELEPSIIKTLSLRYNHIYTIGPLQLLLDQIPEEKKQTGITSLHGYSLVKEEPECFQWLQSKEPNSVVYVNFGSTTVMSLEDMTEFGWGLANSNHYFLWIIRSNLVIGENAVLPPELEEHIKKRGFIASWCSQEKVLKHPSVGGFLTHCGWGSTIESLSAGVPMICWPYSWDQLTNCRYICKEWEVGLEMGTKVKRDEVKRLVQELMGEGGHKMRNKAKDWKEKARIAIAPNGSSSLNIDKMVKEITVLARN

SEQ ID NO: 7 - DNA, 32 bp, Artificial sequence
  RT7 forward primer
attaggatccaatgggaacacaagtaacagag

SEQ ID NO: 8 - DNA, 37 bp, Artificial sequence
  RT7 reverse primer
aatactgcagttaattagtactaatcttacaaaattg

SEQ ID NO: 9 - DNA, 37 bp, Artificial sequence
  RT11 forward primer
attaggatccaatggaagccaagaaaaataaaatgag

SEQ ID NO: 10 - DNA, 35 bp, Artificial sequence
  RT11 reverse primer
aatactgcagtcatttgttgctgcaaagagccatc

SEQ ID NO: 11 - DNA, 34 bp, Artificial sequence
  RT15 forward primer
attaggatccaatggatggttcgaatgaaaagtc

SEQ ID NO: 12 - DNA, 35 bp, Artificial sequence
  RT15 reverse primer
aatactgcagctagacaacatttgatctagtcttg

SEQ ID NO: 13 - DNA, 38 bp, Artificial sequence
  RT18 forward primer
attaggatccaatgagtactactttaaaggtattgatg

SEQ ID NO: 14 - DNA, 36 bp, Artificial sequence
  RT reverse primer
aatactgcagattcacttattactattcctacaaag

SEQ ID NO: 15 - DNA, 31 bp, Artificial sequence
  UGT2_1a forward primer
attaggatccaatggccacttctgactccat

SEQ ID NO: 16 - DNA, 32 bp, Artificial sequence
  UGT2_1a reverse primer
aataaagcttttagctttcgtggtcaatggca

SEQ ID NO: 17 - DNA, 32 bp, Artificial sequence
  UGT85C2 forward primer
attaggatccaatggacgctatggccaccact

SEQ ID NO: 18 - DNA, 32 bp, Artificial sequence
  UGT85C2 reverse primer
aataaagcttttagtttcgagccaagacagtg

SEQ ID NO: 19 - DNA, 1341 bp, Artificial sequence
  RT18 open reading frame optimized for expression in Y. lipolitica
atgtccaccaccctcaaggtcctcatgttccccttcctcgcttacggccacatctctccctacctcaacgttgccaagaagctcgccgaccgaggcttcctcatctacctctgttccacccccatcaacctcaagtccaccatcaacaagatccccgagaagtacgccgactccatccagctcatcgaactccatctccccgagcttcccgagctgcctccccactaccacaccaccaacggtctgcctcccaacctcaaccacatcctccgacgagccctcaagatgtccaagcccaacttctccaagatcatgcagaacctgaagcccgatctgctcatctacgacattctccagcagtgggccgaggatgtcgccaccgagcttaacatccccgccgtcaagctgctcacctctggtgttgctgttttctcttacttcttcaacctcaccaagaagcccgaggtcgagttcccctaccccgctatctacctccgaaagatcgagctggtccgatggtgcgagactctgtccaagcacaacaaggaaggtgaggagcacgacgacggcctcgcctacggcaacatgcagatcatgctcatgtccacttccaagatcctcgaggccaagtacattgactactgcattgagctgaccaactggaaggtcgtccccgtcggctctctcgtccaggactccatcaccaacgacgccgctgacgacgacatggaactcattgactggctcggtactaaggacgagaactccaccgtctttgtctcttttggctccgagtacttcctctccaaagaggacgttgaagaggttgccttcggtctggagctgtccaacgtcaacttcatctgggttgtccgattccccaagggtgaggagaagaacctcgaggacgttctgcccaagggcttcttcgagcgaatcggtgagcgaggccgagtcctcgacaagtttgctccccagccccgaattctcaaccacccctctaccggtggtttcatctctcactgtggctggaactccgccatggagtccattgactttggtgtccccattgtcgccatgcccatgcagctcgaccagcccatgaacgcccgactcattgtcgagcttggtgttgccgtcgagattgtccgagatgatgatggtaagatctaccgaggtgagattgctgagactctcaagggtgtcatcaccggcgagattggtgagatcctccgagccaaggtccgagacatctccaagaacctcaaggccatcaaggacgaggagatggacgttgctgcccaggagctgatccagctctgccgaaactccaataaataa

SEQ ID NO: 20 - DNA, 866 bp, Yarrowia lipolytica
catgctcacttttgttgtcctgatgatctcccgttatttcgccgctcctctggaaaccatccgcccgcaaatcccctctgcccatcttgacaatgcacaatgcatcattctcagcctgcatgaatgcgaaagatggcaatattggtggaggaggcgacggcggtaaacaatggagatagaaaccacaaaagaaacctggaaacccaaaatggactcacgacaactcccccactcccccactccccatctccccctgggcatcagttgcccatcggtatctcaactgtcgcactagttagcgcaaccatcacatactttagacgccaaacaatgggacaactcatcgcgccgaactatgggcagattttaactcgcacaacattaccccaactctaaaaggtaacctcgaccggaaaacgggaagacaggatcagcaaccgtgatcgacagaatcttcagggcactacagttgatagacataggttatgttggtaggtctagacgggcctcggggaattgaccccaccagttgcaagtcacgtgcccctgatacagctagtttagcacatctgcccactacgtctggacgcaccatggtggtgccagtcgcgtgaactcaaacacccactagcctcgggaaggattcagttaaatccgcaccttatttccaacacaaagaagcggttggcggacaaagaacatgtcctttctggggcactgtacattccaggactctgttcaaggtcaaatatacaaaacacagatagagaaacatagacagctgcggccttataaatacctgggcgcacttctctctttttccctcctcatcacacattcgttcaccactaagtcactcgttcaaa

SEQ ID NO: 21 - DNA, 999 bp, Yarrowia lipolytica
ctactgttgctacgattcccccattgcaaccacagtttggggttaccccgcattatattagcatgattacgaaagagataagtatcatatggaacatgtgaagggtagtatgcaggtccggcggagaaagagaatgacgttttcattaagcgattcgcttggcggcttgtgggggatgtgacgatacttacggtaaagaccctgtgtgagagctggtactcgctcgttacttcgctgatctgttgggccgtcaatcgaatctcgtggaacttgcattcttcttaactgtgtctatacaagacacctaatgaaacatacaagctaccgaaatcattttactcgtactgaccggtacggtacttgcacaagtagtgaaacttccgaaaatagccagcctcatgcatcatcgcttcaccccttctgttgacctcaaaagcattccaacggtaaaaaattataacgccgccaactggatggttgtgacggcgttgaccaccaatgtgtgggggctggcggtaggaccgagcttattcgtcccaataagctctttggatttgattctttggggtgtgtggtaaaattcacatggggaagaacacggtggcagtttgaggcagaggcccagcgtgtagttcctagggcatgaatataccgaactcatggcgcagaattgagctgaatgcgcaaaaagctacaggatcaaccgcgttagaaatgccgcaaatgtccactaattccccggactgttccaaatgattctgtggggataaatctcaaactgggttaggctttgtcacgtttctttgtgtcgtgtcggttcgtccggggcaatgtgcccacgcttggctgtctccctacacctcggtaaaaactatcacatgctgcccctctcgagcaagcattaaatgcatatagtcaatctaacgacatatatataggtagggtgcatcctccggtttagctccccagaatatctcttattcattacacaaaaacaac

SEQ ID NO: 22 - DNA, 1000 bp, Yarrowia lipolytica
gtgcaatcacatgttgctactgtacctgctgtggaccacgcacggcggaacgtaccgtacaaatattttcttgctcacatgactctctctcggccgcgcacgccggtggcaaattgctcttgcattggctctgtctctagacgtccaaaccgtccaaagtggcagggtgacgtgatgcgacgcacgaaggagatggcccggtggcgaggaaccggacacggcgagccggcgggaaaaaaggcggaaaacgaaaagcgaagggcacaatctgacggtgcggctgccaccaacccaaggaggctattttgggtcgctttccatttcacattcgccctcaatggccactttgcggtggtgaacatggtttctgaaacaaccccccagaattagagtatattgatgtgtttaagattgggttgctatttggccattgtgggggagggtagcgacgtggaggacattccagggcgaattgagcctagaaagtggtagcattccaaccgtctaagtcgtccgaattgatcgctataactatcacctctctcacatgtctacttccccaaccaacatccccaacctcccccacactaaagttcacgccaataatgtaggcactctttctgggtgtgggacagcagagcaatacggaggggagattacacaacgagccacaattggggagatggtagccatctcactcgacccgtcgacttttggcaacgctcaattacccaccaaatttgggctggagttgaggggaccgtgttccagcgctgtaggaccagcaacacacacggtatcaacagcaaccaacgcccccgctaatgcacccagtactgcgcaggtgtgggccaggtgcgttccagatgcgagttggcgaaccctaagccgacagtgtactttttgggacgggcagtagcaatcgtgggcggaaaccccggtgtatataaaggggtggagaggacggattattagcaccaacacacacacttatactaca

SEQ ID NO: 23 - DNA, 1503 bp, Artificial sequence
  tHMG optimized for expression in Y. lipoltica
atgacccagtctgtgaaggtggttgagaagcacgttcctatcgtcattgagaagcccagcgagaaggaggaggacacctcttctgaagactccattgagctgactgtcggaaagcagcccaagcccgtgaccgagacccgttctctggacgacttggaggctatcatgaaggcaggtaagaccaagctcctggaggaccacgaggttgtcaagctctctctcgaaggcaagctccctttgtatgctcttgagaagcagcttggtgacaacacccgagctgttggcatccgacgatctatcatctcccagcagtctaataccaagactcttgagacctcaaagctcccttacctgcactacgactacgaccgtgtttttggagcctgttgcgagaacgttattggttacatgcctctccccgttggtgttgctggccccatgaacattgatggcaagaactaccacattcctatggccaccactgagggttgtcttgttgcctcaaccatgcgaggttgcaaggccatcaacgccggtggcggtgttaccactgtgcttactcaggacggtatgacacgaggtccttgtgtttccttcccctctctcaagcgggctggagccgctaagatctggcttgattccgaggagggtctcaagtccatgcgaaaggccttcaactccacctctcgatttgctcgtctccagtctcttcactctacccttgctggtaacctgctgtttattcgattccgaaccaccactggtgatgccatgggcatgaacatgatctccaagggcgtcgaacactctctggccgtcatggtcaaggagtacggcttccctgatatggacattgtgtctgtctcgggtaactactgcactgacaagaagcccgcagcgatcaactggatcgaaggccgaggcaagagtgttgttgccgaagccaccatccctgctcacattgtcaagtctgttctcaaaagtgaggttgacgctcttgttgagctcaacatcagcaagaatctgatcggtagtgccatggctggctctgtgggaggtttcaatgcacacgccgcaaacctggtgaccgccatctaccttgccactggccaggatcctgctcagaatgtcgagtcttccaactgcatcacgctgatgagcaacgtcgacggtaacctgctcatctccgtttccatgccttctatcgaggtcggtaccattggtggaggtactattttggagccccagggtgctatgctggagatgcttggcgtgcgaggtcctcacatcgagacccccggtgccaacgcccaacagcttgctcgcatcattgcttctggagttcttgcagcggagctttcgctgtgttctgctcttgctgccggccatcttgtgcaaagtcatatgacccacaaccgttcccaggctcctactccggccaagcagtctcaggccgatctgcagcgtctccaaaacggttcgaatatctgcattcggtcatag

SEQ ID NO: 24 - DNA, 984 bp, Artificial sequence
  GGS optimized for expression in Y. lipolitica
atggattataacagcgcggatttcaaggagatctggggcaaggccgccgacaccgcgctgctgggaccgtacaactacctcgccaacaaccggggccacaacatcagagaacacttgatcgcagcgttcggagcggttatcaaggtggacaagagcgatctcgaaaccatttcgcacatcaccaagattttgcataactcgtcgctgcttgttgatgacgtggaagacaactcgatgctccgacgaggcctgccggcagcccattgtctgtttggagtcccccaaaccatcaactccgccaactacatgtactttgtggctctgcaggaggtgctcaagctcaagtcttatgatgccgtctccattttcaccgaggaaatgatcaacttgcatagaggtcagggtatggatctctactggagagaaacactcacttgcccctcggaagacgagtatctggagatggtggtgcacaagaccggaggactgtttcggctggctctgagacttatgctgtcggtggcatcgaaacaggaggaccatgaaaagatcaactttgatctcacacaccttaccgacacactgggagtcatttaccagattctggatgattacctcaacctgcagtccacggaattgaccgagaacaagggattctgcgaagatatcagcgaaggaaagttttcgtttccgctgattcacagcatccggaccaacccggataaccacgagattctcaacattctcaaacagcgaacaagcgacgcttcactcaaaaagtacgccgtggactacatgagaacagaaaccaagagtttcgactactgcctcaagagaatccaggccatgtcactcaaggcaagttcgtacattgatgatctcgcagcagccggccacgatgtctccaagttgcgagccattttgcattattttgtgtccacctctgactgtgaggagagaaagtactttgaggatgcgcagtga

SEQ ID NO: 25 - DNA, 2232 bp, Artificial sequence
  CPS from S. rebaudiana optimized for expression in Y. lipolitica
atgtgcaaggctgtttccaaggagtactccgatctgctccagaaggacgaggcctctttcaccaagtgggacgacgacaaggtcaaggaccacctcgacaccaacaagaacctctaccccaacgacgagatcaaggagtttgtcgagtccgtcaaggccatgttcggctccatgaacgacggcgagattaatgtctctgcttacgacaccgcctgggttgctctggtccaggatgtcgacggttccggctctcctcagttcccttcctctctcgagtggatcgccaacaaccagctgtccgacggttcttggggtgaccacctgctcttctctgctcacgaccgaatcatcaacaccctggcctgtgtcattgctctgacctcttggaacgtccacccctccaagtgcgagaagggtctgaacttcctccgagagaacatctgcaagctcgaggacgagaacgccgagcacatgcccattggcttcgaggtcaccttcccctctctgattgacattgccaagaagctcaacattgaggtccccgaggacacccccgctctcaaggagatctacgctcgacgagacatcaagctcaccaagatccccatggaggttctccacaaggtccccaccactctcctccactctctcgagggtatgcccgatctcgagtgggagaagctgctcaagctgcagtgcaaggacggctctttcctcttctccccctcttccactgccttcgccctcatgcagaccaaggacgagaagtgtctccagtacctcaccaacattgtcaccaagttcaacggtggtgtccccaacgtctaccccgttgacctctttgagcacatctgggttgttgaccgactccagcgactcggtatcgcccgatacttcaagtccgagatcaaggactgtgtcgagtacatcaacaagtactggaccaagaacggtatctgctgggcccgaaacacccacgtccaggacattgacgacaccgccatgggcttccgagttctgcgagcccacggctacgatgtcacccccgatgtctttcgacagtttgagaaggacggcaagtttgtctgtttcgccggtcagtccacccaggccgtcaccggtatgttcaacgtctaccgagcttctcagatgctcttccccggtgagcgaatcctcgaggacgccaagaagttctcctacaactacctcaaggagaagcagtccaccaacgagctgctcgacaagtggatcattgccaaggatctgcccggtgaggttggctacgccctcgacatcccctggtacgcctctctgccccgactggagactcgatactacctcgagcagtacggtggtgaggacgatgtctggatcggtaagaccctgtaccgaatgggctacgtttccaacaacacctacctcgagatggccaagctcgactacaacaactacgttgccgtcctccagctcgagtggtacaccatccagcagtggtacgtcgacattggtatcgagaagttcgagtccgacaacatcaagtccgtccttgtctcctactacctcgctgctgcctccatcttcgagcccgagcgatccaaggagcgaattgcctgggccaagaccaccatcctcgtcgacaagatcacctccatcttcgactcctcccagtcctccaaggaagatatcaccgccttcattgacaagttccgaaacaagtcctcctccaagaagcactccatcaacggcgagccctggcacgaggtcatggttgctctcaagaaaactctccacggctttgccctcgacgctctgatgacccactctcaggacatccacccccagctccaccaggcctgggagatgtggctcaccaagctccaggacggtgttgatgtcactgctgagctcatggtccagatgatcaacatgaccgccggccgatgggtttccaaggagctcctcacccacccccagtaccagcgactctccactgtcaccaactctgtctgccacgacatcaccaagctccacaacttcaaggagaactccaccaccgtcgactccaaggtccaggagctggtccagctcgttttctccgacacccccgatgatctcgaccaggacatgaagcagaccttcctgactgtcatgaaaactttctactacaaggcctggtgcgaccccaacaccatcaacgaccacatctccaaggtctttgagattgtgatttaa

SEQ ID NO: 26 - DNA, 2274 bp, Artificial sequence
  tKS from S. rebaudiana optimized for expression in Y. lipoltica
atgacctcccacggcggccagaccaaccccaccaacctcatcattgacaccaccaaggagcgaatccagaagcagttcaagaacgtcgagatctccgtttcctcctacgacaccgcctgggtcgccatggtcccctctcccaactcccccaagtctccctgcttccccgagtgtctcaactggctcatcaacaaccagctcaacgacggctcttggggtctggtcaaccacacccacaaccacaaccaccccctcctcaaggactctctctcttccactctcgcctgcattgttgctctcaagcgatggaacgttggcgaggaccagatcaacaagggtctgtctttcattgagtccaacctcgcctccgccaccgagaagtcccagccctcccccattggctttgatatcatcttccccggtctgctcgagtacgccaagaacctcgatatcaacctgctctccaagcagaccgacttctctctcatgctgcacaagcgagagctcgagcagaagcgatgccactccaacgagatggacggctacctggcctacatttccgagggtctgggtaacctctacgactggaacatggtcaagaagtaccagatgaagaacggttccgttttcaactccccctctgccaccgctgctgccttcatcaaccaccagaaccccggctgtctcaactacctcaactctctgctcgacaagtttggtaacgccgtccccactgtctacccccacgatctcttcatccgactctccatggtcgacaccattgagcgactcggtatttcccaccacttccgagtcgagatcaagaacgttctcgatgagacttaccgatgctgggttgagcgagatgagcagatcttcatggacgttgtcacctgtgctctggccttccgactcctccgaatcaacggttacgaggtttcccccgaccccctcgccgagatcaccaacgagctggctctcaaggacgagtacgccgccctcgagacttaccacgcttctcacattctgtaccaagaggatctgtcctccggcaagcagattctcaagtccgccgacttcctcaaggagatcatctccactgactccaaccgactctccaagctcatccacaaggaagtcgagaacgctctcaagttccccatcaacaccggtctggagcgaatcaacacccgacgaaacatccagctctacaacgtcgacaacacccgaattctcaagaccacctaccactcttccaacatctccaacaccgactacctgcgactcgccgtcgaggacttctacacctgccagtccatctaccgagaggagctcaagggtctggagcgatgggttgtcgagaacaagctcgaccagctcaagtttgcccgacaaaagactgcctactgctacttctccgttgctgccaccctctcttctcccgagctctccgacgcccgaatctcttgggccaagaacggtatcctgaccactgttgtcgacgacttctttgacattggtggcaccattgacgagctgaccaacctcatccagtgcgtcgagaagtggaacgtcgacgttgacaaggactgttgttccgagcacgtccgaatcctcttcctggctctcaaggacgccatctgctggatcggtgacgaggccttcaagtggcaggctcgagatgtcacttcccacgtcatccagacctggctcgagctcatgaactccatgctgcgagaggccatctggacccgagatgcctacgtccccaccctcaacgagtacatggagaacgcctacgtcagctttgctctcggtcccattgtcaagcccgccatctactttgtcggtcccaagctgtccgaggagattgtcgagtcctccgagtaccacaacctcttcaagctcatgtccacccagggccgactcctcaacgatatccactccttcaagcgagagttcaaggaaggtaagctcaacgccgttgctctgcacctgtccaacggtgagtccggcaaggtcgaggaagaggtcgtcgaggagatgatgatgatgatcaagaacaagcgaaaggagctcatgaagctcatcttcgaggagaacggctccattgtcccccgagcctgcaaggacgccttctggaacatgtgccacgtcctcaacttcttctacgccaacgacgacggtttcaccggcaacaccattctcgacaccgtcaaggacatcatctacaaccctctggttctggtcaacgagaacgaggagcagaggtaa

SEQ ID NO: 27 - DNA, 1578 bp, Artificial sequence
  KO from Gibberella fujikori optimized for expression in Y. lipolitica
atgtccaagtccaactccatgaactccacctcccacgagactctcttccagcagctcgttctcggcctcgaccgaatgcccctcatggacgtccactggctcatctacgttgcctttggtgcctggctctgctcctacgtcatccacgttctgtcctcttcctccactgtcaaggtccccgtcgtcggttaccgatccgttttcgagcccacctggctcctccgactgcgattcgtctgggagggtggttccatcattggccagggctacaacaagttcaaggactccatcttccaggtccgaaagctcggtaccgacattgtcatcatccctcccaactacattgacgaggtccgaaagctctcccaggacaagacccgatccgtcgagcccttcatcaacgactttgccggccagtacacccgaggtatggtctttctgcagtccgatctccagaaccgagtcatccagcagcgactcacccccaagcttgtctctctcaccaaggtcatgaaggaagagctcgactacgctctgaccaaggagatgcccgacatgaagaacgacgagtgggttgaggtcgacatctcttccatcatggtccgactcatctctcgaatctccgcccgagttttcctcggccccgagcactgccgaaaccaggagtggctcaccaccaccgccgagtactccgagtctctcttcatcaccggcttcatcctccgagttgtcccccacattctccgacccttcattgctcctctgctgccctcttaccgaaccctgctgcgaaacgtttcttccggccgacgagtcattggtgatatcatccgatcccagcagggtgacggtaacgaggacatcctctcttggatgcgagatgctgccactggtgaggagaagcagatcgacaacattgcccagcgaatgctcattctgtctctcgcctccatccacaccaccgccatgaccatgacccacgccatgtacgatctgtgtgcctgccccgagtacattgagcccctccgagatgaggtcaagtccgtcgttggtgcttctggctgggacaagaccgctctcaaccgattccacaagctcgactctttcctcaaggagtcccagcgattcaaccccgttttcctgctcaccttcaaccgaatctaccaccagtccatgaccctctccgatggtaccaacatcccctccggtacccgaattgctgtcccctctcacgccatgctccaggactccgcccacgtccccggtcccactcctcccactgagttcgacggtttccgatactccaagatccgatccgactccaactacgcccagaagtacctcttctccatgaccgactcttccaacatggcctttggctacggtaagtacgcctgccccggccgattctacgcctccaacgagatgaagctgactctggccattctgctcctccagtttgagttcaagctccccgacggtaagggccgaccccgaaacatcaccatcgactccgacatgatccccgacccccgagctcgactctgtgtccgaaagcgatctctgcgtgacgagtaa

SEQ ID NO: 28 - DNA, 1578 bp, Artificial sequence
  KAH_4 optimized for expression in Y. lipolitica
atggagtctctggttgtccacaccgtcaacgccatctggtgcattgtcattgtcggtatcttctccgtcggctaccacgtctacggccgagctgttgtcgagcagtggcgaatgcgacgatctctcaagctccagggtgtcaagggtcctcctccctccatcttcaacggtaacgtttccgagatgcagcgaatccagtccgaggccaagcactgctccggtgacaacatcatctcccacgactactcttcttctctgttcccccactttgaccactggcgaaagcagtacggccgaatctacacctactccactggcctcaagcagcacctctacatcaaccaccccgagatggtcaaggagctctcccagaccaacaccctcaacctcggccgaatcacccacatcaccaagcgactcaaccccattctcggtaacggtatcatcacctccaacggcccccactgggcccaccagcgacgaatcattgcctacgagttcacccacgacaagatcaagggtatggtcggtctgatggtcgagtccgccatgcccatgctcaacaagtgggaggagatggtcaagcgaggtggtgagatgggctgtgacatccgagtcgacgaggacctcaaggatgtctccgctgacgtcattgccaaggcctgtttcggctcttccttctccaagggcaaggccatcttctccatgatccgagatctgctcaccgccatcaccaagcgatccgtcctcttccgattcaacggtttcaccgacatggttttcggctccaagaagcacggtgacgttgacattgacgctctcgagatggagctcgagtcctccatctgggagactgtcaaggagcgagagattgagtgcaaggacacccacaagaaggacctcatgcagctcattctcgagggtgccatgcgatcttgtgacggtaacctgtgggacaagtctgcttaccgacgattcgttgtcgacaactgcaagtccatctactttgccggccacgactccaccgccgtttccgtttcttggtgcctcatgctgctcgctctcaacccctcttggcaggtcaagatccgagatgagattctgtcctcctgcaagaacggtatccccgacgccgagtccatccccaacctcaagaccgtcaccatggtcatccaggagactatgcgactctaccctcccgctcccattgtcggccgagaggcctccaaggacattcgactcggtgatctggttgtccccaagggtgtctgtatctggaccctcatccccgctctgcaccgagatcccgagatctggggtcccgacgccaacgacttcaagcccgagcgattctccgagggtatctccaaggcctgcaagtacccccagtcctacatcccctttggcctcggcccccgaacctgtgtcggcaagaactttggtatgatggaggtcaaggtcctcgtttctctgattgtctccaagttctccttcactctgtctcccacctaccagcactctccctcccacaagctgctcgtcgagccccagcacggtgttgtcatccgagttgtataa

SEQ ID NO: 29 - DNA, 2133 bp, Artificial sequence
  CPR_optimized for expression in Y. lipolitica
atgtcctcctcttcttcttcttccacctccatgattgatctcatggctgccatcatcaagggtgagcccgtcattgtctccgaccccgccaacgcctccgcctacgagtccgttgctgccgagctgtcctccatgctcatcgagaaccgacagtttgccatgatcgtcaccacctccattgctgttctcattggctgcattgtcatgctcgtctggcgacgatctggctccggtaactccaagcgagtcgagcccctcaagcccctggtcatcaagccccgagaagaggagatcgacgacggccgaaagaaggtcaccatcttctttggcacccagaccggtactgctgagggcttcgccaaggctctcggtgaggaagccaaggctcgatacgaaaagacccgattcaagattgtcgacctcgatgattacgctgccgatgacgacgagtacgaggagaagctcaagaaagaggacgttgccttcttcttcctcgccacctacggtgacggtgagcccaccgacaacgctgcccgattctacaagtggttcaccgagggtaacgaccgaggcgagtggctcaagaacctcaagtacggtgttttcggtctgggcaaccgacagtacgagcacttcaacaaggttgccaaggttgtcgacgacatcctcgtcgagcagggtgcccagcgactcgtccaggtcggcctcggtgatgatgaccagtgcatcgaggacgacttcactgcctggcgagaggctctgtggcccgagctcgacaccattctgcgagaggaaggtgacaccgccgttgccaccccctacaccgccgccgtcctcgagtaccgagtctccatccacgactccgaggatgccaagttcaacgacatcaacatggccaacggtaacggctacaccgtctttgacgcccagcacccctacaaggccaacgtcgccgtcaagcgagagctccacacccccgagtccgaccgatcttgtatccacctcgagtttgacattgctggttccggtctgacctacgagactggtgaccacgttggtgtcctctgtgacaacctgtccgagactgtcgacgaggctctgcgactcctcgacatgtcccccgacacttacttctctctgcacgccgagaaagaggacggtactcccatctcttcttctctgccccctcccttccctccctgcaacctgcgaaccgctctgacccgatacgcctgcctcctctcttctcccaagaagtctgctctcgttgctctggccgcccacgcctccgaccccaccgaggctgagcgactcaagcacctcgcctctcccgctggcaaggacgagtactccaagtgggttgtcgagtcccagcgatctctgctcgaggtcatggccgagttcccctccgccaagccccctctcggtgttttcttcgccggtgttgctccccgactccagccccgattctactccatctcctcttcccccaagatcgccgagactcgaatccacgttacctgtgctctggtctacgagaagatgcccaccggccgaatccacaagggtgtctgctccacctggatgaagaacgccgttccctacgagaagtccgagaactgttcctctgctcccatctttgtccgacagtccaacttcaagctcccctccgactccaaggtccccatcatcatgattggccccggtaccggcctcgcccccttccgaggcttcctgcaggagcgactcgccctcgtcgagtccggtgtcgagctcggcccctccgtcctcttctttggctgccgaaaccgacgaatggacttcatctacgaagaggagctccagcgattcgtcgagtccggtgctctcgccgagctctccgttgccttctcccgagagggtcccaccaaggagtacgtccagcacaagatgatggacaaggcctccgacatctggaacatgatctcccagggcgcctacctctacgtctgcggtgacgccaagggtatggcccgagatgtccaccgatctctgcacaccattgcccaggagcagggctccatggactccaccaaggccgagggtttcgtcaagaacctccagacctccggccgatacctccgagatgtctgg

SEQ ID NO: 30 - DNA, 1446 bp, Artificial sequence
  UGT1 optimized for expression in Y. lipolitica
atggacgccatggccaccaccgagaagaagccccacgtcatcttcatccccttccccgcccagtcccacatcaaggccatgctcaagctcgcccagctcctccaccacaagggcctccagatcacctttgtcaacaccgacttcatccacaaccagttcctcgagtcctccggcccccactgtctggacggtgctcccggtttccgatttgagactatccccgatggtgtctcccactcccccgaggcctccatccccatccgagagtctctgctccgatccattgagactaacttcctcgaccgattcattgatctcgtcaccaagctccccgatcctcccacctgtatcatctccgacggtttcctgtccgttttcaccattgatgctgccaagaagctcggtatccccgtcatgatgtactggactctggctgcctgtggtttcatgggtttctaccacatccactctctgatcgagaagggctttgctcctctcaaggacgcctcctacctcaccaacggttacctcgacaccgtcattgactgggtccccggtatggagggtatccgactcaaggacttccccctcgactggtccaccgacctcaacgacaaggttctcatgttcaccaccgaggctccccagcgatcccacaaggtttcccaccacatcttccacaccttcgacgagctcgagccctccatcatcaagactctgtctctgcgatacaaccacatctacaccattggccccctccagctcctcctcgaccagatccccgaggagaagaagcagaccggtatcacctctctgcacggctactctctcgtcaaggaagagcccgagtgcttccagtggctccagtccaaggagcccaactccgttgtctacgtcaactttggctccaccaccgtcatgtctctcgaggacatgaccgagtttggctggggtctggccaactccaaccactacttcctgtggatcatccgatccaacctcgtcattggcgagaacgccgttctgcctcccgagctcgaggagcacatcaagaagcgaggcttcattgcctcttggtgctcccaggagaaggttctcaagcacccctccgtcggtggtttcctgacccactgcggctggggctccaccattgagtctctgtccgctggtgtccccatgatctgctggccctactcctgggaccagctcaccaactgccgatacatctgcaaggagtgggaggttggtctggagatgggtaccaaggtcaagcgagatgaggtcaagcgactcgtccaggagctcatgggcgagggtggtcacaagatgcgaaacaaggccaaggactggaaggagaaggcccgaattgccattgcccccaacggctcttcttctctcaacattgacaagatggtcaaggagatcactgttctcgctcgaaactaa

SEQ ID NO: 31 - DNA, 1419 bp, Artificial sequence
  UGT2 variant optimized for expression in Y. lipolitica
atggccacctccgactccattgttgacgaccgaaagaagctccacattgtcatgttcccctggctcgcctttggccacatcatcccctatctcgagctttccaagctcattgcccagaagggccacaaggtttccttcctctccaccaccaagaacattgaccgactctcctcccacatctctcccctcatcaactttgtcaagctcaccctcccccgagtccaggagctgcccgaggacgccgaggccaccactgatgtccaccccgaggatatcccctacctcaagaaggcctccgacggcctccagcccgaggtcactgagttcctcgagcagcactctcccgactggatcatctacgactacacccactactggctccccgagattgccaagtctctcggtgtctctcgagcccacttctccgtcaccaccccctgggccattgcttacatgggtcccactgccgatgccatgatcaacggttccgactaccgaaccgagcttgaggacttcaccgtccctcccaagtggttccccttccccaccaccgtctgctggcgaaagcacgatctggcccgactcgtcccctacaaggctcccggtatctccgacggttaccgaatgggcctcgtcatcaagggctgcgactgtctgctctccaagacctaccacgagttcggtactcagtggctccgacttctcgaggagctgcaccgagtccccgtcatccccgttggtctgctccctccctccatccccggctctgacaaggacgactcttgggtttccatcaaggagtggctcgacggccaggagaagggctccgttgtctacgttgctctcggttccgaggttctcgtcacccaggaagaggttgtcgagcttgctcacggtctggagctgtccggtctgcccttcttctgggcctaccgaaagcccaagggtcccgccaagtccgactccgtcgagcttcccgatggtttcgtcgagcgagtccgagatcgaggtctggtctggacctcttgggctccccagctccgaatcctctcccacgagtccgttgctggtttcctcacccactgcggttccggctccattgtcgagggcctcatgttcggccaccctctcatcatgctccccatcttcggtgaccagcccctcaacgcccgactccttgaggacaagcaggtcggtatcgagatcccccgaaacgaggaagatggttctttcacccgagactctgttgccgagtctctgcgactcgtcatggtcgaggaagagggtaagatctaccgagagaaggccaaggagatgtccaagctctttggcgacaaggacctccaggaccagtacgtcgacgactttgtcgagtacctccagaagcaccgacgagctgttgccattgaccacgaaagc

SEQ ID NO: 32 - DNA, 1383 bp, Artificial sequence
  UGT3 optimized for expression in Y. lipolitica
atggccgagcagcagaagatcaagaagtctccccacgttctgctcatccccttccctctgcagggccacatcaaccccttcatccagttcggcaagcgactcatctccaagggtgtcaagaccactctggtcaccaccatccacaccctcaactccactctcaaccactccaacaccaccaccacctccatcgagatccaggccatctccgacggctgtgacgagggtggtttcatgtctgctggtgagtcttacctcgagactttcaagcaggtcggttccaagtctctggctgacctcatcaagaagctccagtccgagggtaccaccattgacgccatcatctacgactccatgaccgagtgggttctcgatgtcgccatcgagtttggtattgacggtggctccttcttcacccaggcctgtgtcgtcaactctctctactaccacgtccacaagggtctgatctctctgcccctcggcgagactgtctccgtccccggtttccccgttctgcagcgatgggagactcctctcattctccagaaccacgagcagatccagtccccctggtcccagatgctcttcggccagttcgccaacattgaccaggcccgatgggttttcaccaactccttctacaagctcgaggaagaggtcattgagtggacccgaaagatctggaacctcaaggtcattggccccaccctcccctccatgtacctcgacaagcgactcgatgacgacaaggacaacggtttcaacctctacaaggccaaccaccacgagtgcatgaactggctcgacgacaagcccaaggagtccgttgtctacgttgcctttggctctctggtcaagcacggccccgagcaggttgaggagatcacccgagctctgattgactccgatgtcaacttcctgtgggtcatcaagcacaaggaagagggtaagctccccgagaacctgtccgaggtcatcaagaccggcaagggcctcattgttgcctggtgcaagcagctcgacgttctcgcccacgagtccgtcggctgctttgtcacccactgcggtttcaactccaccctcgaggctatctctctcggtgtccccgttgttgccatgccccagttctccgaccagaccaccaacgccaagctcctcgatgagattctcggtgtcggtgtccgagtcaaggctgacgagaacggtattgtccgacgaggtaacctggcttcttgtatcaagatgatcatggaggaagagcgaggtgtcatcatccgaaagaacgccgtcaagtggaaggatctggccaaggttgctgtccacgagggtggctcttccgacaacgacattgtcgagtttgtctccgagctcatcaaggcctaa

SEQ ID NO: 33 - DNA, 1377 bp, Artificial sequence
  UGT4 optimized for expression in Y. lipolitica
atggagaacaagaccgagactaccgtccgacgacgacgacgaatcattctcttccccgtccccttccagggccacatcaaccccattctgcagctcgccaacgttctgtactccaagggcttctccatcaccatcttccacaccaacttcaacaagcccaagacctccaactacccccacttcactttccgattcatcctcgacaacgacccccaggacgagcgaatctccaacctgcccacccacggtcctctggctggtatgcgaatccccatcatcaacgagcacggtgctgacgagctccgacgagagctcgagctgctcatgctcgcctccgaagaggacgaggaagtctcctgtctgatcaccgatgctctgtggtactttgcccagtccgtcgccgactctctcaacctgcgacgactcgttctcatgacctcctctctgttcaacttccacgcccacgtttctctgccccagtttgacgagctcggttacctcgaccccgatgacaagacccgactcgaggagcaggcttccggtttccccatgctcaaggtcaaggacatcaagtccgcctactccaactggcagattctcaaggagattctcggcaagatgatcaagcagaccaaggcctcctccggtgtcatctggaactccttcaaggagctcgaggagtccgagctcgagactgtcatccgagagatccccgctccctctttcctcatccccctgcccaagcacctcaccgcttcctcctcttctctgctcgaccacgaccgaaccgtctttcagtggctcgaccagcagcccccttcctccgtcctctacgtttccttcggctccacctccgaggtcgacgagaaggacttcctcgagattgctcgaggcctcgttgactccaagcagtccttcctgtgggttgtccgacccggctttgtcaagggctccacctgggttgagcccctgcccgatggtttcctcggtgagcgaggccgaattgtcaagtgggtcccccagcaggaagttctggcccacggtgccattggtgccttctggacccactccggctggaactccactctcgagtccgtctgcgagggtgtccccatgatcttctccgactttggcctcgaccagcccctcaacgcccgatacatgtccgatgttctcaaggtcggtgtctacctcgagaacggctgggagcgaggtgagattgccaacgccatccgacgagtcatggtcgacgaggaaggtgagtacatccgacagaacgcccgagtcctcaagcagaaggccgatgtctctctcatgaagggtggttcttcttacgagtctctcgagtctctcgtttcctacatctcttctttgtaa

SEQ ID NO: 34 - DNA, 1419 bp, Artificial sequence
  UGT2 variant optimized for expression in Y. lipolitica
atggctacttccgactccattgtcgacgaccgaaagaagctccacattgtcatgttcccctggctcgcctttggccacatcattccctacctcgagctttccaagctcattgcccagaagggccacaaggtttctttcctctccaccaccaagaacattgaccgactctcctcccacatctctcctctcatcaacgttgtccagctcaccctcccccgagtccaggagctgcccgaggacgccgaggccaccaccgatgtccaccccgaggatatcccctacctcaagaaggcctccgacggtctgcagcccgaggtcaccgagttcctcgagcagcactctcccgactggatcatctacgactacacccactactggctcccctccattgccaccaagcacggtgtctctcgagcccacttctccgtcaccaccccctgggccattgcctacatgggccccactgctgacgccatgatcaacggttccgatggccgaaccacccccgaggacttcactgtccctcccaagtggttccccttccccaccaaggtctgctggcgaaagcacgatctggcccgactcgttccctacaaggcccccggtatctccgacggctaccgaatgggtctggtcatcaagggctgcgactgtctgctctccaagacctaccacgagtttggcacccagtggctccgactcctcgagactctccaccgaaagcccgtcatccccgtcggtctgctccctccctccatccccggctccgacaaggacgactcttgggtttccatcaaggagtggctcgacggccaggagaagggctctgttgtctacgttgctctcggttccgaggttctcgtcacccaggacgaggttgttgagctggcccacggtctggagctgtccggcctccccttcgtctgggcttaccgaaaccccaagggtcccgccaagtccgactccgtcgagcttcccgatggtttcgtcgagcgagtccgagatcgaggtctggtctggacctcttgggctccccagctccgaatcctctcccacgagtccgtctgtggtttcctcacccactgcggttccggctccatcgtcgagggtctgatgttcggccaccccctcatcatgctccccatcttcggtgaccagcccctcaacgcccgactccttgaggacaagcaggtcggtatcgagatcccccgaaacgaagaggacggttccttcacccgagactctgttgctgagtctctccgactcgtcatggtcgaggaagagggtaagatctaccgagagaaggccaaggagatgtccaagctgttcggtgacaaggatctccaggaccagtacgtcgacgactttgtcgagtacctccagaagcaccgacgagctgttgccattgaccacgagtct

(19) United States
(12) Patent Application Publication
(10) Pub. No.: US 2018/0057850 A1
(43) Pub. Date: Mar. 1, 2018

(54) UDP-GLYCOSYLTRANSFERASES FROM SOLANUM LYCOPERSICUM

(71) Applicant: DSM IP ASSETS B.V., Heerlen (NL)

(72) Inventors: Hendrik Jan BOSCH, Wageningen (NL); Martinus Julius BEEKWILDER, Wageningen (NL); Viktor Marius BOER, Echt (NL)

(21) Appl. No.: 15/560,318

(22) PCT Filed: Mar. 23, 2016
(86) PCT No.: PCT/EP2016/056449

§ 371 Date: Sep. 21, 2017

Related U.S. Application Data
(60) Provisional application No. 62/136,759, filed on Mar. 23, 2015.

(51) Int. Cl. C12P 19/56; C12N 9/02; C12N 9/10; A23L 27/30; A23L 2/60

(52) U.S. Cl. CPC C12P 19/56; C12N 9/0042; C12Y 106/02004; A23V 2002/00; C12Y 204/01017; A23L 27/36; A23L 2/60; C12N 9/1051

(57) ABSTRACT

The present invention relates to polypeptides having UDP-Glycosyltransferase activity derived from Solanum lycopersicum and having the amino acid sequence set out in any of SEQ ID NO: 1 to 4 or an amino acid sequence having at least about 30% sequence identity thereto. The application also relates to recombinant hosts comprising a recombinant nucleic acid sequence encoding said polypeptides and uses thereof prepare glycosylated diterpenes, like steviol glycoside. The host cells might comprise further enzymes of the steviol glycoside biosynthesis pathway.

### FIELD OF THE INVENTION

The present invention relates to a recombinant host comprising a recombinant nucleic acid sequence encoding a UDP-glycosyltransferase (UGT) polypeptide. The invention also relates to a process for the preparation of a glycosylated diterpene using such a recombinant host and to a fermentation broth which may be the result of such a process. The invention further relates to a glycosylated diterpene obtained by such a process or obtainable from such a fermentation broth and to a composition comprising two or more such glycosylated diterpenes. In addition the invention relates to a foodstuff, feed or beverage which comprises such a glycosylated diterpene or a such composition. The invention also relates to a method for converting a first glycosylated diterpene into a second glycosylated diterpene using the above-mentioned recombinant host.

### BACKGROUND TO THE INVENTION

The leaves of the perennial herb, Stevia rebaudiana Bert., accumulate quantities of intensely sweet compounds known as steviol glycosides. Whilst the biological function of these compounds is unclear, they have commercial significance as alternative high potency sweeteners.

These sweet steviol glycosides have functional and sensory properties that appear to be superior to those of many high potency sweeteners. In addition, studies suggest that stevioside can reduce blood glucose levels in Type II diabetics and can reduce blood pressure in mildly hypertensive patients.

Steviol glycosides accumulate in Stevia leaves where they may comprise from 10 to 20% of the leaf dry weight. Stevioside and rebaudioside A are both heat and pH stable and suitable for use in carbonated beverages and many other foods. Stevioside is between 110 and 270 times sweeter than sucrose, rebaudioside A between 150 and 320 times sweeter than sucrose. In addition, rebaudioside D is also a high-potency diterpene glycoside sweetener which accumulates in Stevia leaves. It may be about 200 times sweeter than sucrose. Rebaudioside M is a further high-potency diterpene glycoside sweetener. It is present in trace amounts in certain stevia variety leaves, but has been suggested to have a superior taste profile.

Steviol glycosides have traditionally been extracted from the Stevia plant. In Stevia, (−)-kaurenoic acid, an intermediate in gibberellic acid (GA) biosynthesis, is converted into the tetracyclic diterpene steviol, which then proceeds through a multi-step glycosylation pathway to form the various steviol glycosides. However, yields may be variable and affected by agriculture and environmental conditions. Also, Stevia cultivation requires substantial land area, a long time prior to harvest, intensive labour and additional costs for the extraction and purification of the glycosides.

More recently, interest has grown in producing steviol glycosides using fermentative processes. WO2013/110673 and WO2015/007748 describe microorganisms that may be used to produce at least the steviol glycosides rebaudioside A and rebaudioside D.

Further improvement of such microoganisms is desirable in order that higher amounts of steviol glycosides may be produced and/or additional or new steviol glycosides and/or higher amounts of specific steviol glycosides and/or mixtures of steviol glycosides having desired ratios of different steviol glycosides.

### SUMMARY OF THE INVENTION

In Stevia rebaudiana, steviol is synthesized from GGPP, which is formed by the deoxyxylulose 5-phosphate pathway. The activity of two diterpene cyclases (−)-copalyl diphosphate synthase (CPS) and (−)-kaurene synthase (KS) results in the formation of (−)-Kaurene which is then oxidized in a three step reaction by (−)-kaurene oxidase (KO) to form (−)-kaurenoic acid.

In Stevia rebaudiana leaves, (−)-kaurenoic acid is then hydroxylated, by ent-kaurenoic acid 13-hydroxylase (KAH) to form steviol. Steviol is then glycosylated by a series of UDP-glycosyltransferases (UGTs) leading to the formation of a number of steviol glycosides. Specifically, these molecules can be viewed as a steviol molecule, with its carboxyl hydrogen atom replaced by a glucose molecule to form an ester, and an hydroxyl hydrogen with combinations of glucose and rhamnose to form an acetal.

These pathways may be reconstructed in recombinant hosts, for example yeasts such as yeasts of the genera Saccharomyces and Yarrowia.

The invention relates to the identification of polypeptides having UDP-glycosyltransferase (UGT), typically having improved properties in comparison to those that are currently known. These polypeptides may be used to generate recombinant hosts that produce higher amounts of steviol glycosides and/or additional or new steviol glycosides and/or higher amounts of specific steviol glycosides and/or mixtures of steviol glycosides having desired ratios of different steviol glycosides.

Thus, the invention also relates to a recombinant host capable of producing a glycosylated diterpene, i.e. a diterpene glycoside such as a steviol glycoside, for example steviolmonoside, steviolbioside, stevioside, rebaudioside A, rebaudioside B, rebaudioside C, rebaudioside D, rebaudioside E, rebaudioside F, rebaudioside M, rubusoside, dulcoside A, steviol-13-monoside, steviol-19-monoside or 13-[(β-D-Glucopyranosyl)oxy)kaur-16-en-18-oic acid 2-O-β-D-glucopyranosyl-β-D-glucopyranosyl ester steviol-19-diside.

Accordingly, the invention relates to a recombinant host comprising a recombinant nucleic acid sequence, typically having UDP-glycosyltransferase (UGT) activity such as UGT2 activity, encoding a polypeptide having:
a. the amino acid sequence set forth in SEQ ID NO: 1 or an amino acid sequence having at least about 30% sequence identity thereto;
b. the amino acid sequence set forth in SEQ ID NO: 2 or an amino acid sequence having at least about 30% sequence identity thereto;
c. the amino acid sequence set forth in SEQ ID NO: 3 or an amino acid sequence having at least about 30% sequence identity thereto; or
d. the amino acid sequence set forth in SEQ ID NO: 4 or an amino acid sequence having at least about 30% sequence identity thereto.

The invention also relates to:
a process for the preparation of a glycosylated diterpene which comprises fermenting a recombinant host of the invention in a suitable fermentation medium, and optionally recovering the glycosylated diterpene;
a fermentation broth comprising a glycosylated diterpene obtainable by the process of the invention;
a glycosylated diterpene obtained by such a process or obtainable from such a fermentation broth;
a composition comprising two or more such diterpenes;
a foodstuff, feed or beverage which comprises such a glycosylated diterpene; and
a method for converting a first glycosylated diterpene into a second glycosylated diterpene, which method comprises:
   contacting said first glycosylated diterpene with a recombinant host of the invention, a cell free extract derived from such a recombinant host or an enzyme preparation derived from either thereof;
   thereby to convert the first glycosylated diterpene into the second glycosylated diterpene.

### BRIEF DESCRIPTION OF THE DRAWINGS

FIG. 1 sets out Western blot detection of His-tagged UGTs

FIG. 2 sets out Western blot of UGT2_1a and RT18. Lanes 1,2,3,4: 0.5, 1.0, 1.9, 3.8 μg of UGT2_1a crude enzyme extract. Lane 5 and 6: 31.9 and 63.8 μg RT18 crude enzyme extract.

FIG. 3 sets out the effect of the expression of RT18 on the production of RebM

FIG. 4 sets out the effect of the expression of RT18 on the production of RebD

FIG. 5 sets out a schematic diagram of the potential pathways leading to biosynthesis of steviol glycosides.

FIG. 6 sets out a schematic diagram of the potential pathways leading to biosynthesis of steviol glycosides. The compound shown with an asterisk is 13-[(β-D-Glucopyranosyl)oxy)kaur-16-en-18-oic acid 2-O-β-D-glucopyranosyl-β-D-glucopyranosyl ester.

### DESCRIPTION OF THE SEQUENCE LISTING

A description of the sequences is set out in Table 10. Sequences described herein may be defined with reference to the sequence listing or with reference to the database accession numbers also set out herein, for example in Table 10.

### DETAILED DESCRIPTION OF THE INVENTION

Throughout the present specification and the accompanying claims, the words “comprise”, “include” and “having” and variations such as “comprises”, “comprising”, “includes” and “including” are to be interpreted inclusively. That is, these words are intended to convey the possible inclusion of other elements or integers not specifically recited, where the context allows.

The articles “a” and “an” are used herein to refer to one or to more than one (i.e. to one or at least one) of the grammatical object of the article. By way of example, “an element” may mean one element or more than one element.

Herein, “rebaudioside” may be shortened to “reb”. That is to say, rebaudioside A and reb A, for example, are intended to indicate the same molecule.

The term “recombinant” when used in reference to a cell, nucleic acid, protein or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all. The term “recombinant” is synonymous with “genetically modified”.

The invention concerns polypeptides identified as having UDP-glycosyltransferase (UGT) activity which can be used in recombinant hosts, typically for the production of diterpene glycosides, such as steviol glycosides.

For the purposes of this invention, a polypeptide having UGT activity is one which has glycosyltransferase activity (EC 2.4), i.e. that can act as a catalyst for the transfer of a monosaccharide unit from an activated nucleotide sugar (also known as the “glycosyl donor”) to a glycosyl acceptor molecule, usually an alcohol. The glycosyl donor for a UGT is typically the nucleotide sugar uridine diphosphate glucose (uracil-diphosphate glucose, UDP-glucose). A polypeptide suitable for use in a host of the invention typically has UGT activity and a polynucleotide sequence as described herein typically encodes such a polypeptide. Typically, the polypeptides for use in a host of the invention are polypeptides having UGT2-type activity.

The invention thus provides a recombinant host comprising a recombinant nucleic acid sequence encoding a polypeptide comprising:
a. the amino acid sequence set forth in SEQ ID NO: 1 or an amino acid sequence having at least about 30% sequence identity thereto;
b. the amino acid sequence set forth in SEQ ID NO: 2 or an amino acid sequence having at least about 30% sequence identity thereto;
c. the amino acid sequence set forth in SEQ ID NO: 3 or an amino acid sequence having at least about 30% sequence identity thereto; or
d. the amino acid sequence set forth in SEQ ID NO: 4 or an amino acid sequence having at least about 30% sequence identity thereto.

The polypeptide encoded by the recombinant nucleic acid sequence typically has UGT activity, such as UGT2 activity. A recombinant host of the invention is typically capable of producing a glycosylated diterpene, for example a steviol glycoside.

A polypeptide encoded by a recombinant nucleic acid present in a recombinant host of the invention may comprise an amino acid sequence having at least about 35%, at least about 40%, at least about 50%, at least about 60%, at least about 65%, at least about 70%, at least about 75%, at least about 80%, at least about 85%, at least about, 86%, at least about 87%, at least about 88%, at least about 89%, at least about 90%, at least about 91%, at least about 92%, at least about 93%, at least about 94%, at least about 95%, at least about 96%, at least about 97%, at least about 98% or at least about 99% sequence identity to any one of SEQ ID NOs: 1, 2, 3 or 4.

Thus, the invention relates to:
a recombinant host comprising a recombinant nucleic acid sequence encoding a polypeptide, typically having UGT activity, which comprises an amino acid sequence having at least about 35%, at least about 40%, at least about 50%, at least about 60%, at least about 65%, at least about 70%, at least about 75%, at least about 80%, at least about 85%, at least about, 86%, at least about 87%, at least about 88%, at least about 89%, at least about 90%, at least about 91%, at least about 92%, at least about 93%, at least about 94%, at least about 95%, at least about 96%, at least about 97%, at least about 98% or at least about 99% sequence identity to SEQ ID NO: 1;
a recombinant host comprising a recombinant nucleic acid sequence encoding a polypeptide, typically having UGT activity, which comprises an amino acid sequence having at least about 35%, at least about 40%, at least about 50%, at least about 60%, at least about 65%, at least about 70%, at least about 75%, at least about 80%, at least about 85%, at least about, 86%, at least about 87%, at least about 88%, at least about 89%, at least about 90%, at least about 91%, at least about 92%, at least about 93%, at least about 94%, at least about 95%, at least about 96%, at least about 97%, at least about 98% or at least about 99% sequence identity to SEQ ID NO: 2;
a recombinant host comprising a recombinant nucleic acid sequence encoding a polypeptide, typically having UGT activity, which comprises an amino acid sequence having at least about 35%, at least about 40%, at least about 50%, at least about 60%, at least about 65%, at least about 70%, at least about 75%, at least about 80%, at least about 85%, at least about, 86%, at least about 87%, at least about 88%, at least about 89%, at least about 90%, at least about 91%, at least about 92%, at least about 93%, at least about 94%, at least about 95%, at least about 96%, at least about 97%, at least about 98% or at least about 99% sequence identity ao to SEQ ID NO: 3;
a recombinant host comprising a recombinant nucleic acid sequence encoding a polypeptide, typically having UGT activity, which comprises an amino acid sequence having at least about 35%, at least about 40%, at least about 50%, at least about 60%, at least about 65%, at least about 70%, at least about 75%, at least about 80%, at least about 85%, at least about, 86%, at least about 87%, at least about 88%, at least about 89%, at least about 90%, at least about 91%, at least about 92%, at least about 93%, at least about 94%, at least about 95%, at least about 96%, at least about 97%, at least about 98% or at least about 99% sequence identity to SEQ ID NO: 4.

As used herein, the term “polypeptide” refers to a molecule comprising amino acid residues linked by peptide bonds and containing more than five amino acid residues. The amino acids are identified by either the single-letter or three-letter designations. The term “protein” as used herein is synonymous with the term “polypeptide” and may also refer to two or more polypeptides. Thus, the terms “protein”, “peptide” and “polypeptide” can be used interchangeably. Polypeptides may optionally be modified (e.g., glycosylated, phosphorylated, acylated, farnesylated, prenylated, sulfonated, and the like) to add functionality. Polypeptides exhibiting activity may be referred to as enzymes. It will be understood that, as a result of the degeneracy of the genetic code, a multitude of nucleotide sequences encoding a given polypeptide may be produced.

The term “nucleic acid sequence” (or “”polynucleotide“) as used in the present invention refers to a nucleotide polymer including at least 5 nucleotide units. A nucleic acid refers to a ribonucleotide polymer (RNA), deoxynucleotide polymer (DNA) or a modified form of either type of nucleic acid or synthetic form thereof or mixed polymers of any of the above. Nucleic acids may include either or both naturally-occurring and modified nucleic acids linked together by naturally-occurring and/or non-naturally occurring nucleic acid linkages. The nucleic acid molecules may be modified chemically or biochemically or may contain non-natural or derivatized nucleic acid bases, as will be readily appreciated by those of skill in the art. Such modifications include, for example, labels, methylation, substitution of one or more of the naturally occurring nucleic acids with an analog, internucleotide modifications such as uncharged linkages (e.g., methyl phosphonates, phosphotriesters, phosphoramidates, carbamates, etc.), charged linkages (e.g., phosphorothioates, phosphorodithioates, etc.), pendent moieties (e.g., polypeptides), intercalators (e.g., acridine, psoralen, etc.), chelators, alkylators, and modified linkages (e.g., alpha anomeric nucleic acids, etc.) The term nucleic acid is also intended to include any topological conformation, including single-stranded (sense strand and antisense strand), double-stranded, partially duplexed, triplex, hairpinned, circular and padlocked conformations. Also included are synthetic molecules that mimic nucleic acids in their ability to bind to a designated sequence via hydrogen bonding and other chemical interactions. Such molecules are known in the art and include, for example, those in which peptide linkages substitute for phosphate linkages in the backbone of the molecule. A reference to a nucleic acid sequence encompasses its complement unless otherwise specified. Thus, a reference to a nucleic acid molecule having a particular sequence should be understood to encompass its complementary strand, with its complementary sequence. The complementary strand is also useful, e.g., for antisense therapy, hybridization probes and PCR primers. The term “nucleic acid”, “polynucleotide” and “polynucleotide sequence” can be used interchangeably herein.A polypeptide encoded by a recombinant nucleic acid for use in a recombinant host of the invention may comprise a signal peptide and/or a propeptide sequence. In the event that a polypeptide comprises a signal peptide and/or a propeptide, sequence identity may be calculated over the mature polypeptide sequence.

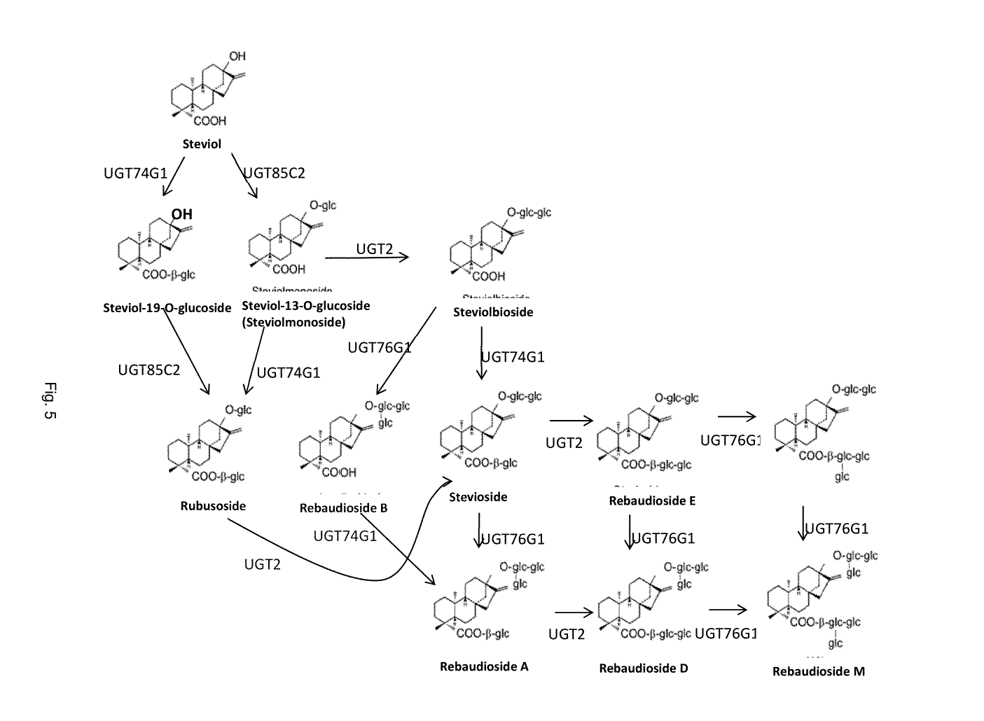

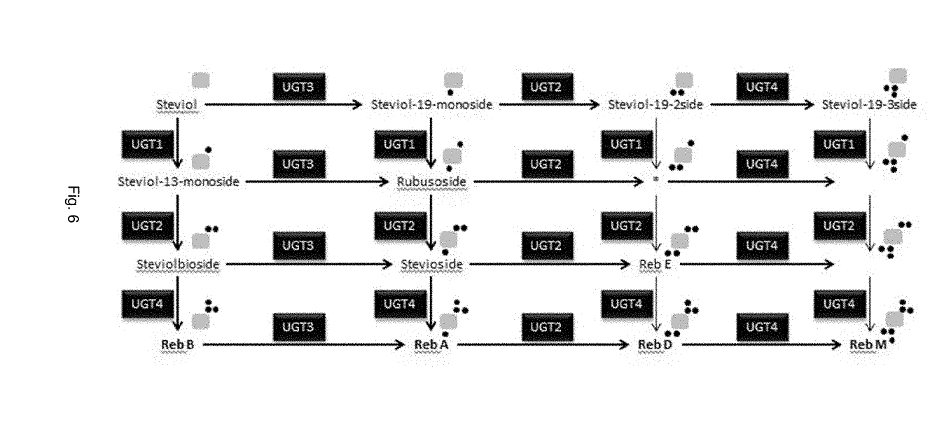

The polypeptide typically has UGT activity and more preferably has UGT2 activity. FIGS. 5 and 6 illustrate a non-exhaustive list of reactions that may be catalyzed by a polypeptide having UGT2 activity.

A polypeptide having UGT2 activity is one which may function as a uridine 5′-diphospho glucosyl: steviol-13-O-glucoside transferase (also referred to as a steviol-13-monoglucoside 1,2-glucosylase), transferring a glucose moiety to the C-2′ of the 13-O-glucose of the acceptor molecule, steviol-13-O-glucoside. Typically, a suitable UGT2 polypeptide may also function as a uridine 5′-diphospho glucosyl: rubusoside transferase transferring a glucose moiety to the C-2′ of the 13-O-glucose of the acceptor molecule, rubusoside. That is to say be capable of converting steviol-13-monoside to steviolbioside and/or capable of converting rubusoside to stevioside.

A polypeptide having UGT2 activity may also or alternatively catalyze reactions that utilize steviol glycoside substrates other than steviol-13-O-glucoside and rubusoside, e.g., a functional UGT2 polypeptide may utilize stevioside as a substrate, transferring a glucose moiety to the C-2′ of the 19-O-glucose residue to produce rebaudioside E. A functional UGT2 polypeptide may also or alternatively utilize rebaudioside A as a substrate, transferring a glucose moiety to the C-2′ of the 19-O-glucose residue to produce rebaudioside D.

A polypeptide having UGT2 activity may also catalyze reactions that utilize steviol-19-glucoside or rubusoside as a substrate, e.g., a functional UGT2 polypeptide may utilize steviol-19-glucoside or rubusoside as a substrate, transferring a glucose moiety to the 19 position to produce steviol-19-2side or 13-[(β-D-Glucopyranosyl)oxy)kaur-16-en-18-oic acid 2-O-β-D-glucopyranosyl-β-D-glucopyranosyl ester respectively.

However, a functional UGT2 polypeptide typically does not transfer a glucose moiety to steviol compounds having a 1,3-bound glucose at the C-13 position, i.e., transfer of a glucose moiety to steviol 1,3-bioside and 1,3-stevioside typically does not occur.

A polypeptide having UGT2 activity may also or alternatively transfer sugar moieties from donors other than uridine diphosphate glucose. For example, a polypeptide having UGT2 activity act as a uridine 5′-diphospho D-xylosyl: steviol-13-O-glucoside transferase, transferring a xylose moiety to the C-2′ of the 13-O-glucose of the acceptor molecule, steviol-13 -O-glucoside. As another example, a polypeptide having UGT2 activity may act as a uridine 5′-diphospho L-rhamnosyl: steviol-13-O-glucoside transferase, transferring a rhamnose moiety to the C-2′ of the 13-O-glucose of the acceptor molecule, steviol.

One or more of the above-described activities may be used to define a polypeptide having UGT2 activity encoded by a recombinant nucleic acid sequence for use in a recombinant host of the invention. Such a polypeptide may have improved UGT2 activity in respect of one or more of the above-described activities in comparison with the UGT2_1a polypeptide (SEQ ID NO: 6).

A polynucleotide encoding a polypeptide for use in a recombinant host of the invention may be used to steer production of steviol glycosides in a recombinant cell to a desired steviol glycoside, such as rebaudioside A, rebaudioside D or rebaudioside M. For example, a UGT2 polypeptide which preferentially catalyzes conversion of steviol-13-monoside to steviolbioside and/or conversion of rubusoside to stevioside may help to steer production towards rebaudiosideA, whereas a UGT2 polypeptide which preferentially catalyzes conversion of stevioside to rebE or rubusoside to a compound with an additional sugar at the 19 position may help to steer production towards rebaudioside M. That is to say preference for addition of a sugar moiety at the 13 position may help steer production towards rebaudioside A, whereas preference for addition of a sugar moiety at the 19 position may help steer production towards rebaudioside M.

A recombinant nucleic acid sequence for use in a recombinant host of the invention may be provided in the form of a nucleic acid construct. The term “nucleic acid construct” refers to as a nucleic acid molecule, either single-or double-stranded, which is isolated from a naturally occurring gene or which has been modified to contain segments of nucleic acid which are combined and juxtaposed in a manner which would not otherwise exist in nature. The term nucleic acid construct is synonymous with the term “expression cassette” when the nucleic acid construct contains all the control sequences required for expression of a coding sequence, wherein said control sequences are operably linked to said coding sequence.

A recombinant nucleic acid sequence for use in a recombinant host of the invention may be provided in the form of an expression vector, wherein the polynucleotide sequence is operably linked to at least one control sequence for the expression of the polynucleotide sequence in a recombinant host cell.

The term “operably linked” as used herein refers to two or more nucleic acid sequence elements that are physically linked and are in a functional relationship with each other. For instance, a promoter is operably linked to a coding sequence if the promoter is able to initiate or regulate the transcription or expression of a coding sequence, in which case the coding sequence should be understood as being “under the control of” the promoter. Generally, when two nucleic acid sequences are operably linked, they will be in the same orientation and usually also in the same reading frame. They usually will be essentially contiguous, although this may not be required.

An expression vector comprises a polynucleotide coding for a polypeptide as described herein, operably linked to the appropriate control sequences (such as a promoter, and transcriptional and translational stop signals) for expression and/or translation in vitro, or in the host cell of the polynucleotide.

The expression vector may be any vector (e.g., a plasmid or virus), which can be conveniently subjected to recombinant DNA procedures and can bring about the expression of the polynucleotide. The choice of the vector will typically depend on the compatibility of the vector with the cell into which the vector is to be introduced. The vectors may be linear or closed circular plasmids. The vector may be an autonomously replicating vector, i.e., a vector, which exists as an extra-chromosomal entity, the replication of which is independent of chromosomal replication, e.g., a plasmid, an extra-chromosomal element, a mini-chromosome, or an artificial chromosome.

Alternatively, the vector may be one which, when introduced into the host cell, is integrated into the genome and replicated together with the chromosome(s) into which it has been integrated. The integrative cloning vector may integrate at random or at a predetermined target locus in the chromosomes of the host cell. A vector may comprise one or more selectable markers, which permit easy selection of transformed cells.

Standard genetic techniques, such as overexpression of enzymes in the host cells, as well as for additional genetic modification of host cells, are known methods in the art, such as described in Sambrook and Russel (2001) “Molecular Cloning: A Laboratory Manual (3^{rd} edition), Cold Spring Harbor Laboratory, Cold Spring Harbor Laboratory Press, or F. Ausubel et al, eds., “Current protocols in molecular biology”, Green Publishing and Wiley Interscience, New York (1987). Methods for transformation and genetic modification of fungal host cells are known from e.g. EP-A-0 635 574, WO 98/46772, WO 99/60102 and WO 00/37671.

A recombinant host of the invention may comprise any polypeptide as described herein. Typically, a recombinant host of the invention is capable of producing a glycosylated diterpene, such as a steviol glycoside. For example, a recombinant host of the invention may be capable of producing one or more of, for example, steviol-13-monoside, steviol-19-monoside, 13-[(β-D-Glucopyranosyl)oxy)kaur-16-en-18-oic acid 2-O-β-D-glucopyranosyl-β-D-glucopyranosyl ester, rubusoside, stevioside, steviol-19-diside, steviolbioside, rebA, rebE, rebD or rebM.

Thus, a recombinant host of the invention will typically comprise polynucleotides encoding polypeptides having UGT1, UGT2, UGT2 and UGT4 activity and polypeptides which provide for the production of steviol in the host (which may then be converted to one or more steviol glycosides).

One polynucleotide may encode more than one of such polypeptides. One polynucleotide may encode a polypeptide having more than one of the activities UGT1, UGT2, UGT3 or UGT4 or the activity of a polypeptide providing for production of steviol in the host. Accordingly, a recombinant host according to the invention may comprise one or more recombinant nucleotide sequence(s) encoding one of more of:
a polypeptide having ent-copalyl pyrophosphate synthase activity;
a polypeptide having ent-Kaurene synthase activity;
a polypeptide having ent-Kaurene oxidase activity; and
a polypeptide having kaurenoic acid 13-hydroxylase activity.

A recombinant host may comprise one or more recombinant polynucleotide sequences encoding all four such polypeptides.

For the purposes of this invention, a polypeptide having ent-copalyl pyrophosphate synthase (EC 5.5.1.13) is capable of catalyzing the chemical reation:

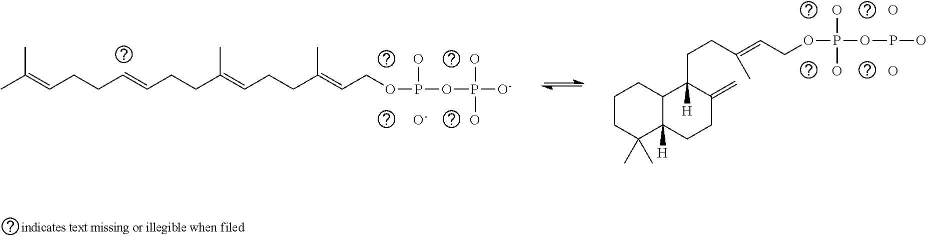

This enzyme has one substrate, geranylgeranyl pyrophosphate, and one product, ent-copalyl pyrophosphate. This enzyme participates in gibberellin biosynthesis. This enzyme belongs to the family of isomerase, specifically the class of intramolecular lyases. The systematic name of this enzyme class is ent-copalyl-diphosphate lyase (decyclizing). Other names in common use include having ent-copalyl pyrophosphate synthase, ent-kaurene synthase A, and ent-kaurene synthetase A.

Suitable nucleic acid sequences encoding an ent-copalyl pyrophosphate synthase may for instance comprise a sequence as set out in SEQ ID. NO: 1, 3, 5, 7, 17, 19, 59, 61, 141, 142, 151, 152, 153, 154, 159, 160, 182 or 184 of WO2015/007748.

For the purposes of this invention, a polypeptide having ent-kaurene synthase activity (EC 4.2.3.19) is a polypeptide that is capable of catalyzing the chemical reaction:

ent-copalyl diphosphate [symbol] ent-kaurene+ diphosphate

Hence, this enzyme has one substrate, ent-copalyl diphosphate, and two products, ent-kaurene and diphosphate.

This enzyme belongs to the family of lyases, specifically those carbon-oxygen lyases acting on phosphates. The systematic name of this enzyme class is ent-copalyl-diphosphate diphosphate-lyase (cyclizing, ent-kaurene-forming). Other names in common use include ent-kaurene synthase B, ent-kaurene synthetase B, ent-copalyl-diphosphate diphosphate-lyase, and (cyclizing). This enzyme participates in diterpenoid biosynthesis.

Suitable nucleic acid sequences encoding an ent-Kaurene synthase may for instance comprise a sequence as set out in SEQ ID. NO: 9, 11, 13, 15, 17, 19, 63, 65, 143, 144, 155, 156, 157, 158, 159, 160, 183 or 184 of WO2015/007748.

ent-copalyl diphosphate synthases may also have a distinct ent-kaurene synthase activity associated with the same protein molecule. The reaction catalyzed by ent-kaurene synthase is the next step in the biosynthetic pathway to gibberellins. The two types of enzymic activity are distinct, and site-directed mutagenesis to suppress the ent-kaurene synthase activity of the protein leads to build up of ent-copalyl pyrophosphate.

Accordingly, a single nucleotide sequence used in a recombinant host of the invention may encode a polypeptide having ent-copalyl pyrophosphate synthase activity and ent-kaurene synthase activity. Alternatively, the two activities may be encoded two distinct, separate nucleotide sequences.

For the purposes of this invention, a polypeptide having ent-kaurene oxidase activity (EC 1.14.13.78) is a polypeptide which is capable of catalysing three successive oxidations of the 4-methyl group of ent-kaurene to give kaurenoic acid. Such activity typically requires the presence of a cytochrome P450.

Suitable nucleic acid sequences encoding an ent-Kaurene oxidase may for instance comprise a sequence as set out in SEQ ID. NO: 21, 23, 25, 67, 85, 145, 161, 162, 163, 180 or 186 of WO2015/007748.

For the purposes of the invention, a polypeptide having kaurenoic acid 13-hydroxylase activity (EC 1.14.13) is one which is capable of catalyzing the formation of steviol (ent-kaur-16-en-13-ol-19-oic acid) using NADPH and O₂. Such activity may also be referred to as ent-ka 13-hydroxylase activity.

Suitable nucleic acid sequences encoding a kaurenoic acid 13-hydroxylase may for instance comprise a sequence as set out in SEQ ID. NO: 27, 29, 31, 33, 69, 89, 91, 93, 95, 97, 146, 164, 165, 166, 167 or 185 of WO2015/007748.

A recombinant host of the invention may comprise a recombinant nucleic acid sequence encoding a polypeptide having NADPH-cytochrome p450 reductase activity. That is to say, a recombinant host of the invention may be capable of expressing a nucleotide sequence encoding a polypeptide having NADPH-cytochrome p450 reductase activity. For the purposes of the invention, a polypeptide having NADPH-Cytochrome P450 reductase activity (EC 1.6.2.4; also known as NADPH:ferrihemoprotein oxidoreductase, NADPH:hemoprotein oxidoreductase, NADPH:P450 oxidoreductase, P450 reductase, POR, CPR, CYPOR) is typically one which is a membrane-bound enzyme allowing electron transfer to cytochrome P450 in the microsome of the eukaryotic cell from a FAD-and FMN-containing enzyme NADPH:cytochrome P450 reductase (POR; EC 1.6.2.4).

A recombinant host of the invention may comprise one or more recombinant nucleic acid sequences encoding one or more UGT polypeptides, in addition to RT7, RT11, RT15 or RT18 or related sequences as described herein. Such additional UGTs may be selected so as to produce a desired diterpene glycoside, such as a steviol glycoside. Schematic diagrams of steviol glycoside formation are set out in Humphrey et al., Plant Molecular Biology (2006) 61: 47-62 and Mohamed et al., J. Plant Physiology 168 (2011) 1136-1141. In addition, FIGS. 5 and 6 sets out a schematic diagram of steviol glycoside formation.

A recombinant host of the invention may thus comprise one or more recombinant nucleic acid sequences encoding one or more of:

(i) a polypeptide having UGT74G1 activity (UGT3 activity);

(ii) a polypeptide having UGT85C2 activity (UGT1 activity); and

(iii) a polypeptide having UGT76G1 activity (UGT4 activity).

FIGS. 5 and 6 set out schematic diagram of the potential pathways leading to biosynthesis of steviol glycosides.

A recombinant host of the invention will typically comprise at least one recombinant nucleic acid encoding a polypeptide having UGT1 activity, at least one recombinant nucleic acid encoding a polypeptide having UGT2 activity, at least one recombinant nucleic acid encoding a polypeptide having UGT3 activity and at least one recombinant nucleic acid encoding a polypeptide having UGT4 activity. One nucleic acid may encode two or more of such polypeptides.

A recombinant host of the invention typically comprises polynucleotides expressing at least one of each of a UGT1, UGT2, UGT3 and UGT4 polypeptide and a polypeptide having ent-copalyl pyrophosphate synthase activity, a polypeptide having ent-Kaurene synthase activity, a polypeptide having ent-Kaurene oxidase activity and a polypeptide having kaurenoic acid 13-hydroxylase activity. In such a recombinant host, all polynucleotides encoding such polypeptides may be recombinant.

A nucleic acid encoding a polypeptide as described herein may be used to steer production of steviol glycosides in a recombinant cell to a desired steviol glycoside, such as rebaudioside A, rebaudioside D or rebaudioside M. For example, a recombinant nucleic acid which encodes a UGT2 polypeptide which preferentially catalyzes conversion of steviol-13-monoside to steviolbioside and/or conversion of rubusoside to stevioside may help to steer production towards rebaudiosideA, whereas a recombinant nucleic acid which encodes a UGT2 polypeptide which preferentially catalyzes conversion of stevioside to rebE or rubusoside to a compound with an additional sugar at the 19 position may help to steer production towards rebaudioside M. That is to say preference for addition of a sugar moiety at the 13 position may help steer production towards rebaudioside A, whereas preference for addition of a sugar moiety at the 19 position may help steer production towards rebaudioside M.A recombinant host of the invention may comprises a nucleotide sequence encoding a polypeptide capable of catalyzing the addition of a C-13-glucose to steviol. That is to say, a recombinant host of the invention may comprise a UGT which is capable of catalyzing a reaction in which steviol is converted to steviolmonoside.

Such a recombinant host of the invention may comprise a nucleotide sequence encoding a polypeptide having the activity shown by UDP-glycosyltransferase (UGT) UGT85C2, whereby the nucleotide sequence upon transformation of the host confers on that host the ability to convert steviol to steviolmonoside.

UGT85C2 activity is transfer of a glucose unit to the 13-OH of steviol. Thus, a suitable UGT85C2 may function as a uridine 5′-diphospho glucosyl: steviol 13-OH transferase, and a uridine 5′-diphospho glucosyl: steviol-19-0-glucoside 13-OH transferase. A functional UGT85C2 polypeptides may also catalyze glucosyl transferase reactions that utilize steviol glycoside substrates other than steviol and steviol-19-O-glucoside. Such sequences may be referred to as UGT1 sequences herein.

A recombinant host of the invention may comprises a nucleotide sequence encoding a polypeptide having UGT activity may comprise a nucleotide sequence encoding a polypeptide capable of catalyzing the addition of a C-19-glucose to steviolbioside and/or to rebaudioside B. That is to say, a recombinant host of the invention may comprise a UGT which is capable of catalyzing a reaction in which steviolbioside is converted to stevioside and/or in which rebaudioside B is converted to rebaudioside A. Accordingly, such a recombinant host may be capable of converting steviolbioside to stevioside and/or rebaudioside B is converted to rebaudioside A. Expression of such a nucleotide sequence may confer on the recombinant host the ability to produce at least stevioside and/or rebaudioside A.

A recombinant host of the invention may thus also comprise a nucleotide sequence encoding a polypeptide having the activity shown by UDP-glycosyltransferase (UGT) UGT74G1, whereby the nucleotide sequence upon transformation of the host confers on the cell the ability to convert steviolbioside to stevioside.

Suitable UGT74G1 polypeptides may be capable of transferring a glucose unit to the 13-OH or the 19-COOH, respectively, of steviol. A suitable UGT74G1 polypeptide may function as a uridine 5′-diphospho glucosyl: steviol19-COOH transferase and a uridine 5′-diphospho glucosyl: steviol-13-O-glucoside 19-COOH transferase. Functional UGT74G1 polypeptides also may catalyze glycosyl transferase reactions that utilize steviol glycoside substrates other than steviol and steviol-13-O-glucoside, or that transfer sugar moieties from donors other than uridine diphosphate glucose. Such sequences may be referred to herein as UGT3 sequences.

A recombinant host of the invention may comprise a nucleotide sequence encoding a polypeptide capable of catalyzing glucosylation of the C-3′ of the glucose at the C-13 position of stevioside. That is to say, a recombinant host of the invention may comprise a UGT which is capable of catalyzing a reaction in which stevioside is converted to rebaudioside A. Accordingly, such a recombinant host may be capable of converting stevioside to rebaudioside A. Expression of such a nucleotide sequence may confer on the host the ability to produce at least rebaudioside A.

A recombinant host of the invention may thus also comprise a nucleotide sequence encoding a polypeptide having the activity shown by UDP-glycosyltransferase (UGT) UGT76G1, whereby the nucleotide sequence upon transformation of a host confers on that host the ability to convert stevioside to rebaudioside A and/or steviolbioside to rebaudioside B.

A suitable UGT76G1 adds a glucose moiety to the C-3′of the C-13-O-glucose of the acceptor molecule, a steviol 1,2 glycoside. Thus, UGT76G1 functions, for example, as a uridine 5′-diphosphoglucosyl: steviol 13-0-1,2 glucoside C-3 ‘ glucosyl transferase and a uridine 5’-diphospho glucosyl: steviol-19-0-glucose, 13-0-1,2 bioside C-3′ glucosyl transferase. Functional UGT76G1 polypeptides may also catalyze glucosyl transferase reactions that utilize steviol glycoside substrates that contain sugars other than glucose, e.g., steviol rhamnosides and steviol xylosides. Such sequences may be referred to herein as UGT4 sequences. A UGT4 may alternatively or in addition be capable of converting RebD to RebM.

A recombinant host of the invention typically comprises nucleotide sequences encoding polypeptides having all four UGT activities described above. A given nucleic acid may encode a polypeptide having one or more of the above activities. For example, a nucleic acid encode for a polypeptide which has two, three or four of the activities set out above. Preferably, a recombinant host of the invention comprises UGT1, UGT2 and UGT3 and UGT4 activity. Suitable UGT1, UGT3 and UGT4 sequences are described in in Table 1 of WO2015/007748.

A recombinant host of the invention may comprise a recombinant nucleic acid sequence encoding an additional polypeptide having UGT2 activity. That is to say, a recombinant host of the invention may comprise a nucleic acid sequence encoding a variant UGT2 of the invention and one or more additional, different, variant of the invention or any another, different, UGT2.

Use of a nucleic acid sequence encoding a RT7, RT11, RT15 or RT18 polypeptide (or related polypeptide as described herein) may be useful in improving rebA production in a recombinant host of the invention.

Use of a nucleic acid sequence encoding a RT7, RT11, RT15 or RT18 polypeptide (or related polypeptide as described herein) may be useful in improving rebM production in a recombinant host of the invention.

In a recombinant host of the invention, the ability of the host to produce geranylgeranyl diphosphate (GGPP) may be upregulated. Upregulated in the context of this invention implies that the recombinant host produces more GGPP than an equivalent non-recombinant host.

Accordingly, a recombinant host of the invention may comprise one or more nucleotide sequence(s) encoding hydroxymethylglutaryl-CoA reductase, farnesyl-pyrophosphate synthetase and geranylgeranyl diphosphate synthase, whereby the nucleotide sequence(s) upon transformation of a host confer(s) on that host the ability to produce elevated levels of GGPP. Thus, a recombinant host according to the invention may comprise one or more recombinant nucleic acid sequence(s) encoding one or more of hydroxymethylglutaryl-CoA reductase, farnesyl-pyrophosphate synthetase and geranylgeranyl diphosphate synthase.

Accordingly, a recombinant host of the invention may comprise nucleic acid sequences encoding one or more of:

a polypeptide having hydroxymethylglutaryl-CoA reductase activity;

a polypeptide having farnesyl-pyrophosphate synthetase activity;

a polypeptide having geranylgeranyl diphosphate synthase activity.

A recombinant host of the invention may be, for example, an multicellular organism or a cell thereof or a unicellular organism. A host of the invention may be a prokaryotic, archaebacterial or eukaryotic host cell.

A prokaryotic host cell may, but is not limited to, a bacterial host cell. An eukaryotic host cell may be, but is not limited to, a yeast, a fungus, an amoeba, an algae, an animal, an insect or a plant host cell.

An eukaryotic host cell may be a fungal host cell. “Fungi” include all species of the subdivision Eumycotina (Alexopoulos, C. J., 1962, In: Introductory Mycology, John Wiley & Sons, Inc., New York). The term fungus thus includes among others filamentous fungi and yeast.

“Filamentous fungi” are herein defined as eukaryotic microorganisms that include all filamentous forms of the subdivision Eumycotina and Oomycota (as defined by Hawksworth et al., 1995, supra). The filamentous fungi are characterized by a mycelial wall composed of chitin, cellulose, glucan, chitosan, mannan, and other complex polysaccharides. Vegetative growth is by hyphal elongation and carbon catabolism is obligatory aerobic. Filamentous fungal strains include, but are not limited to, strains of Acremonium, Aspergillus, Agaricus, Aureobasidium, Cryptococcus, Corynascus, Chrysosporium, Filibasidium, Fusarium, Humicola, Magnaporthe, Monascus, Mucor, Myceliophthora, Mortierella, Neocallimastix, Neurospora, Paecilomyces, Penicillium, Piromyces, Phanerochaete Podospora, Pycnoporus, Rhizopus, Schizophyllum, Sordaria, Talaromyces, Rasmsonia, Thermoascus, Thielavia, Tolypocladium, Trametes and Trichoderma. Preferred filamentous fungal strains that may serve as host cells belong to the species Aspergillus niger, Aspergillus oryzae, Aspergillus fumigatus, Penicillium chrysogenum, Penicillium citrinum, Acremonium chrysogenum, Trichoderma reesei, Rasamsonia emersonii (formerly known as Talaromyces emersonii), Aspergillus sojae, Chrysosporium lucknowense, Myceliophtora thermophyla. Reference host cells for the comparison of fermentation characteristics of transformed and untransformed cells, include e.g. Aspergillus niger CBS120.49, CBS 513.88, Aspergillus oryzae ATCC16868, ATCC 20423, IFO 4177, ATCC 1011, ATCC 9576, ATCC14488-14491, ATCC 11601, ATCC12892, Aspergillus fumigatus AF293 (CBS101355), P. chrysogenum CBS 455.95, Penicillium citrinum ATCC 38065, Penicillium chrysogenumP2, Acremonium chrysogenum ATCC 36225, ATCC 48272, Trichoderma reesei ATCC 26921, ATCC 56765, ATCC 26921, Aspergillus sojae ATCC11906, Chrysosporium lucknowense ATCC44006 and derivatives of all of these strains. Particularly preferred as filamentous fungal host cell are Aspergillus niger CBS 513.88 and derivatives thereof.

An eukaryotic host cell may be a yeast cell. Preferred yeast host cells may be selected from the genera: Saccharomyces (e.g., S. cerevisiae, S. bayanus, S. pastorianus, S. carlsbergensis), Brettanomyces, Kluyveromyces, Candida (e.g., C. krusei, C. revkaufi, C. pulcherrima, C. tropicalis, C. utilis), lssatchenkia (eg. l. orientalis) Pichia (e.g., P. pastoris), Schizosaccharomyces, Hansenula, Kloeckera, Pachysolen, Schwanniomyces, Trichosporon, Yarrowia (e.g., Y. lipolytica (formerly classified as Candida lipolytica)), Yamadazyma.

Prokaryotic host cells may be bacterial host cells. Bacterial host cell may be Gram negative or Gram positive bacteria. Examples of bacteria include, but are not limited to, bacteria belonging to the genus Bacillus (e.g., B. subtilis, B. amyloliquefaciens, B. licheniformis, B. puntis, B. megaterium, B. halodurans, B. pumilus,), Acinetobacter, Nocardia, Xanthobacter, Escherichia (e.g., E. coli (e.g., strains DH 1 OB, Stbl2, DH5-alpha, DB3, DB3.1), DB4, DBS, JDP682 and ccdA-over (e.g., U.S. application Ser. No. 09/518,188))), Streptomyces, Erwinia, Klebsiella, Serratia (e.g., S. marcessans), Pseudomonas (e.g., P. aeruginosa), Salmonella (e.g., S. typhimurium, S. typhi). Bacteria also include, but are not limited to, photosynthetic bacteria (e.g., green non-sulfur bacteria (e.g., Choroflexus bacteria (e.g., C. aurantiacus), Chloronema (e.g., C. gigateum )), green sulfur bacteria (e.g., Chlorobium bacteria (e.g., C. limicola), Pelodictyon (e.g., P. luteolum), purple sulfur bacteria (e.g., Chromatium (e.g., C. okenii)), and purple non-sulfur bacteria (e.g., Rhodospirillum (e.g., R. rubrum), Rhodobacter (e.g. R. sphaeroides, R. capsulatus), and Rhodomicrobium bacteria (e.g., R. vanellii)).

Host cells may be host cells from non-microbial organisms. Examples of such cells, include, but are not limited to, insect cells (e.g., Drosophila (e.g., D. melanogaster), Spodoptera (e.g., S. frugiperda Sf9 or Sf21 cells) and Trichoplusa (e.g., High-Five cells); nematode cells (e.g., C. elegans cells); avian cells; amphibian cells (e.g., Xenopus laevis cells); reptilian cells; and mammalian cells (e.g., NIH3T3, 293, CHO, COS, VERO, C127, BHK, Per-C6, Bowes melanoma and HeLa cells).

A recombinant host according to the present invention may be able to grow on any suitable carbon source known in the art and convert it to a glycosylated diterpene, eg. a steviol glycoside. The recombinant host may be able to convert directly plant biomass, celluloses, hemicelluloses, pectines, rhamnose, galactose, fucose, maltose, maltodextrines, ribose, ribulose, or starch, starch derivatives, sucrose, lactose and glycerol. Hence, a preferred host expresses enzymes such as cellulases (endocellulases and exocellulases) and hemicellulases (e.g. endo-and exo-xylanases, arabinases) necessary for the conversion of cellulose into glucose monomers and hemicellulose into xylose and arabinose monomers, pectinases able to convert pectines into glucuronic acid and galacturonic acid or amylases to convert starch into glucose monomers. Preferably, the host is able to convert a carbon source selected from the group consisting of glucose, xylose, arabinose, sucrose, lactose and glycerol. The host cell may for instance be a eukaryotic host cell as described in WO03/062430, WO06/009434, EP1499708B1, WO2006096130 or WO04/099381.

Thus, in a further aspect, the invention also provides a process for the preparation of a glycosylated diterpene which comprises fermenting a recombinant host of the invention which is capable of producing at least one glycosylated diterpene in a suitable fermentation medium, and optionally recovering the glycosylated diterpene.

The fermentation medium used in the process for the production of a glycosylated diterpene may be any suitable fermentation medium which allows growth of a particular eukaryotic host cell. The essential elements of the fermentation medium are known to the person skilled in the art and may be adapted to the host cell selected.

Preferably, the fermentation medium comprises a carbon source selected from the group consisting of plant biomass, celluloses, hemicelluloses, pectines, rhamnose, galactose, fucose, fructose, maltose, maltodextrines, ribose, ribulose, or starch, starch derivatives, sucrose, lactose, fatty acids, triglycerides and glycerol. Preferably, the fermentation medium also comprises a nitrogen source such as ureum, or an ammonium salt such as ammonium sulphate, ammonium chloride, ammoniumnitrate or ammonium phosphate.

The fermentation process according to the present invention may be carried out in batch, fed-batch or continuous mode. A separate hydrolysis and fermentation (SHF) process or a simultaneous saccharification and fermentation (SSF) process may also be applied. A combination of these fermentation process modes may also be possible for optimal productivity. A SSF process may be particularly attractive if starch, cellulose, hemicelluose or pectin is used as a carbon source in the fermentation process, where it may be necessary to add hydrolytic enzymes, such as cellulases, hemicellulases or pectinases to hydrolyse the substrate.

The recombinant host used in the process for the preparation of a glycosylated diterpene may be any suitable recombinant host as defined herein above. It may be advantageous to use a recombinant eukaryotic recombinant host according to the invention in the process since most eukaryotic cells do not require sterile conditions for propagation and are insensitive to bacteriophage infections. In addition, eukaryotic host cells may be grown at low pH to prevent bacterial contamination.

The recombinant host according to the present invention may be a facultative anaerobic microorganism. A facultative anaerobic recombinant host can be propagated aerobically to a high cell concentration. This anaerobic phase can then be carried out at high cell density which reduces the fermentation volume required substantially, and may minimize the risk of contamination with aerobic microorganisms.

The fermentation process for the production of a glycosylated diterpene according to the present invention may be an aerobic or an anaerobic fermentation process.

An anaerobic fermentation process may be herein defined as a fermentation process run in the absence of oxygen or in which substantially no oxygen is consumed, preferably less than 5, 2.5 or 1 mmol/L/h, and wherein organic molecules serve as both electron donor and electron acceptors. The fermentation process according to the present invention may also first be run under aerobic conditions and subsequently under anaerobic conditions.

The fermentation process may also be run under oxygen-limited, or micro-aerobical, conditions. Alternatively, the fermentation process may first be run under aerobic conditions and subsequently under oxygen-limited conditions. An oxygen-limited fermentation process is a process in which the oxygen consumption is limited by the oxygen transfer from the gas to the liquid. The degree of oxygen limitation is determined by the amount and composition of the ingoing gasflow as well as the actual mixing/mass transfer properties of the fermentation equipment used.

The production of a glycosylated diterpene in the process according to the present invention may occur during the growth phase of the host cell, during the stationary (steady state) phase or during both phases. It may be possible to run the fermentation process at different temperatures.

The process for the production of a glycosylated diterpene may be run at a temperature which is optimal for the recombinant host. The optimum growth temperature may differ for each transformed recombinant host and is known to the person skilled in the art. The optimum temperature might be higher than optimal for wild type organisms to grow the organism efficiently under non-sterile conditions under minimal infection sensitivity and lowest cooling cost. Alternatively, the process may be carried out at a temperature which is not optimal for growth of the recombinant host.

The process for the production of a glycosylated diterpene according to the present invention may be carried out at any suitable pH value. If the recombinant host is a yeast, the pH in the fermentation medium preferably has a value of below 6, preferably below 5,5, preferably below 5, preferably below 4,5, preferably below 4, preferably below pH 3,5 or below pH 3,0, or below pH 2,5, preferably above pH 2. An advantage of carrying out the fermentation at these low pH values is that growth of contaminant bacteria in the fermentation medium may be prevented.

Such a process may be carried out on an industrial scale. The product of such a process is one or more glycosylated diterpenes, such as one or more steviol glycosides.

Recovery of glycosylated diterpene(s) from the fermentation medium may be performed by known methods in the art, for instance by distillation, vacuum extraction, solvent extraction, or evaporation.

In the process for the production of a glycosylated diterpene according to the invention, it may be possible to achieve a concentration of above 5 mg/I fermentation broth, preferably above 10 mg/I, preferably above 20 mg/l, preferably above 30 mg/I fermentation broth, preferably above 40 mg/I, more preferably above 50 mg/I, preferably above 60 mg/I, preferably above 70, preferably above 80 mg/I, preferably above 100 mg/I, preferably above 1 g/l, preferably above 5 g/l, preferably above 10 g/l, for example above 20 g/l, but usually up to a concentration of about 200 g/l, such as up to about 150 g/l, such as up to about 100 g/l, for example up to about 70 g/l. Such concentrations may be concentration of the total broth or of the supernatant.

The invention further provides a fermentation broth comprising a glycosylated diterpene obtainable by the process of the invention for the preparation of a glycosylated diterpene.

In the event that one or more glycosylated diterpenes is expressed within a recombinant host of the invention, such cells may need to be treated so as to release them. Preferentially, at least one glycosylated diterpene, such as a steviol glycoside, for example rebA or rebM, is produced extracellularly

The invention also provides a glycosylated diterpene obtained by a process according to the invention for the preparation of a glycosylated diterpene or obtainable from a fermentation broth of the invention. Such a glycosylated diterpene may be a non-naturally occurring glycosylated diterpene, that is to say one which is not produced in plants.

Also provided is a composition comprising one or more steviol glycosides obtainable by process for the preparation of a glycosylated diterpene or obtainable from a fermentation broth of the invention. Such a composition may comprise two or more glycosylated diterpenes obtainable by a process of the invention for the preparation of a glycosylated diterpene or obtainable from a fermentation broth of the invention. In such a composition, one or more of the glycosylated diterpenes may be a non-naturally occurring glycosylated diterpene, that is to say one which is not produced in plants.

Furthermore, the invention provides a method for converting a first glycosylated diterpene into a second glycosylated diterpene, which method comprises:
contacting said first glycosylated diterpene with a recombinant host of the invention, a cell free extract derived from such a recombinant host or an enzyme preparation derived from either thereof;
thereby to convert the first glycosylated diterpene into the second glycosylated diterpene.

In such a method, the second glycosylated diterpene may be steviol-19-diside, steviolbioside, stevioside, 13-[(β-D-Glucopyranosyl)oxy)kaur-16-en-18-oic acid 2-O-β-D-glucopyranosyl-β-D-glucopyranosyl ester, RebE or RebD.

In such a method, the first glycosylated diterpene may be steviol-13-monoside, steviol-19-monoside, rubusoside, stevioside, rebaudioside A or 13-[(β-D-Glucopyranosyl)oxy)kaur-16-en-18-oic acid 2-O-β-D-glucopyranosyl-β-D-glucopyranosyl ester and the second glycosylated diterpene is steviol-19-diside, steviolbioside, stevioside, 13-[(β-D-Glucopyranosyl)oxy)kaur-16-en-18-oic acid 2O-β-D-glucopyranosyl-β-D-glucopyranosyl ester, RebE or RebD.

These are the first and second steviol glycosides in relation to a reaction catalysed by a polypeptide described herein having UGT2 activity.

That is to say, the invention relates to a method of bioconversion or biotransformation.

A steviol glycoside or composition produced by the fermentation process according to the present invention may be used in any application known for such compounds. In particular, they may for instance be used as a sweetener, for example in a food or a beverage. According to the invention therefore, there is provided a foodstuff, feed or beverage which comprises a glycosylated diterpene, such as a steviol glycoside, or a composition of the invention.

For example a glycosylated diterpene or a composition of the invention may be formulated in soft drinks, as a tabletop sweetener, chewing gum, dairy product such as yoghurt (eg. plain yoghurt), cake, cereal or cereal-based food, nutraceutical, pharmaceutical, edible gel, confectionery product, cosmetic, toothpastes or other oral cavity composition, etc. In addition, a glycosylated diterpene or a composition of the invention can be used as a sweetener not only for drinks, foodstuffs, and other products dedicated for human consumption, but also in animal feed and fodder with improved characteristics.

Accordingly, the invention provides, inter alia, a foodstuff, feed or beverage which comprises a diterpene or glycosylated diterpene prepared according to a process of the invention.

During the manufacturing of foodstuffs, drinks, pharmaceuticals, cosmetics, table top products, chewing gum the conventional methods such as mixing, kneading, dissolution, pickling, permeation, percolation, sprinkling, atomizing, infusing and other methods can be used.

The glycosylated diterpene, for example a steviol glycoside, or a composition of the invention can be used in dry or liquid forms. It can be added before or after heat treatment of food products. The amount of the sweetener depends on the purpose of usage. It can be added alone or in the combination with other compounds.

Compounds produced according to the method of the invention may be blended with one or more further non-caloric or caloric sweeteners. Such blending may be used to improve flavour or temporal profile or stability. A wide range of both non-caloric and caloric sweeteners may be suitable for blending with a glycosylated diterpene or a composition of the invention. For example, non-caloric sweeteners such as mogroside, monatin, aspartame, acesulfame salts, cyclamate, sucralose, saccharin salts or erythritol. Caloric sweeteners suitable for blending with a glycosylated diterpene or a composition of the invention include sugar alcohols and carbohydrates such as sucrose, glucose, fructose and HFCS. Sweet tasting amino acids such as glycine, alanine or serine may also be used.

A glycosylated diterpene or a composition of the invention can be used in the combination with a sweetener suppressor, such as a natural sweetener suppressor. It may be combined with an umami taste enhancer, such as an amino acid or a salt thereof.

A glycosylated diterpene or a composition of the invention can be combined with a polyol or sugar alcohol, a carbohydrate, a physiologically active substance or functional ingredient (for example a carotenoid, dietary fiber, fatty acid, saponin, antioxidant, nutraceutical, flavonoid, isothiocyanate, phenol, plant sterol or stanol (phytosterols and phytostanols), a polyols, a prebiotic, a probiotic, a phytoestrogen, soy protein, sulfides/thiols, amino acids, a protein, a vitamin, a mineral, and/or a substance classified based on a health benefits, such as cardiovascular, cholesterol-reducing or anti-inflammatory.

A composition with a glycosylated diterpene or a composition of the invention may include a flavoring agent, an aroma component, a nucleotide, an organic acid, an organic acid salt, an inorganic acid, a bitter compound, a protein or protein hydrolyzate, a surfactant, a flavonoid, an astringent compound, a vitamin, a dietary fiber, an antioxidant, a fatty acid and/or a salt.

A glycosylated diterpene or a composition of the invention may be applied as a high intensity sweetener to produce zero calorie, reduced calorie or diabetic beverages and food products with improved taste characteristics. Also it can be used in drinks, foodstuffs, pharmaceuticals, and other products in which sugar cannot be used.

In addition, a glycosylated diterpene or a composition of the invention may be used as a sweetener not only for drinks, foodstuffs, and other products dedicated for human consumption, but also in animal feed and fodder with improved characteristics.

The examples of products where a glycosylated diterpene or a composition of the invention can be used as a sweetening compound can be as alcoholic beverages such as vodka, wine, beer, liquor, sake, etc; natural juices, refreshing drinks, carbonated soft drinks, diet drinks, zero calorie drinks, reduced calorie drinks and foods, yogurt drinks, instant juices, instant coffee, powdered types of instant beverages, canned products, syrups, fermented soybean paste, soy sauce, vinegar, dressings, mayonnaise, ketchups, curry, soup, instant bouillon, powdered soy sauce, powdered vinegar, types of biscuits, rice biscuit, crackers, bread, chocolates, caramel, candy, chewing gum, jelly, pudding, preserved fruits and vegetables, fresh cream, jam, marmalade, flower paste, powdered milk, ice cream, sorbet, vegetables and fruits packed in bottles, canned and boiled beans, meat and foods boiled in sweetened sauce, agricultural vegetable food products, seafood, ham, sausage, fish ham, fish sausage, fish paste, deep fried fish products, dried seafood products, frozen food products, preserved seaweed, preserved meat, tobacco, medicinal products, and many others. In principal it can have unlimited applications.

The sweetened composition comprises a beverage, non-limiting examples of which include non-carbonated and carbonated beverages such as colas, ginger ales, root beers, ciders, fruit-flavored soft drinks (e.g., citrus-flavored soft drinks such as lemon-lime or orange), powdered soft drinks, and the like; fruit juices originating in fruits or vegetables, fruit juices including squeezed juices or the like, fruit juices containing fruit particles, fruit beverages, fruit juice beverages, beverages containing fruit juices, beverages with fruit flavorings, vegetable juices, juices containing vegetables, and mixed juices containing fruits and vegetables; sport drinks, energy drinks, near water and the like drinks (e.g., water with natural or synthetic flavorants); tea type or favorite type beverages such as coffee, cocoa, black tea, green tea, oolong tea and the like; beverages containing milk components such as milk beverages, coffee containing milk components, cafe au lait, milk tea, fruit milk beverages, drinkable yogurt, lactic acid bacteria beverages or the like; and dairy products.

Generally, the amount of sweetener present in a sweetened composition varies widely depending on the particular type of sweetened composition and its desired sweetness. Those of ordinary skill in the art can readily discern the appropriate amount of sweetener to put in the sweetened composition.

A glycosylated diterpene or a composition of the invention can be used in dry or liquid forms. It can be added before or after heat treatment of food products. The amount of the sweetener depends on the purpose of usage. It can be added alone or in the combination with other compounds.

During the manufacturing of foodstuffs, drinks, pharmaceuticals, cosmetics, table top products, chewing gum the conventional methods such as mixing, kneading, dissolution, pickling, permeation, percolation, sprinkling, atomizing, infusing and other methods can be used.

Thus, compositions of the present invention can be made by any method known to those skilled in the art that provide homogenous even or homogeneous mixtures of the ingredients. These methods include dry blending, spray drying, agglomeration, wet granulation, compaction, co-crystallization and the like.

In solid form a glycosylated diterpene or a composition of the invention can be provided to consumers in any form suitable for delivery into the comestible to be sweetened, including sachets, packets, bulk bags or boxes, cubes, tablets, mists, or dissolvable strips. The composition can be delivered as a unit dose or in bulk form.

For liquid sweetener systems and compositions convenient ranges of fluid, semi-fluid, paste and cream forms, appropriate packing using appropriate packing material in any shape or form shall be invented which is convenient to carry or dispense or store or transport any combination containing any of the above sweetener products or combination of product produced above.

The composition may include various bulking agents, functional ingredients, colorants, flavors.

The terms “sequence homology” or “sequence identity” or “homology” or “identity” are used interchangeably herein. For the purpose of this invention, it is defined here that in order to determine the percentage of sequence homology or sequence identity of two amino acid sequences or of two nucleic acid sequences, the sequences are aligned for optimal comparison purposes. In order to optimize the alignment between the two sequences gaps may be introduced in any of the two sequences that are compared. Such alignment can be carried out over the full length of the sequences being compared. Alternatively, the alignment may be carried out over a shorter length, for example over about 20, about 50, about 100 or more nucleic acids/based or amino acids. The sequence identity is the percentage of identical matches between the two sequences over the reported aligned region.

A comparison of sequences and determination of percentage of sequence identity between two sequences can be accomplished using a mathematical algorithm. The skilled person will be aware of the fact that several different computer programs are available to align two sequences and determine the identity between two sequences (Kruskal, J. B. (1983) An overview of sequence comparison In D. Sankoff and J. B. Kruskal, (ed.), Time warps, string edits and macromolecules: the theory and practice of sequence comparison, pp. 1-44 Addison Wesley). The percent sequence identity between two amino acid sequences or between two nucleotide sequences may be determined using the Needleman and Wunsch algorithm for the alignment of two sequences. (Needleman, S. B. and Wunsch, C. D. (1970) J. Mol. Biol. 48, 443-453). Both amino acid sequences and nucleotide sequences can be aligned by the algorithm. The Needleman-Wunsch algorithm has been implemented in the computer program NEEDLE. For the purpose of this invention the NEEDLE program from the EMBOSS package was used (version 2.8.0 or higher, EMBOSS: The European Molecular Biology Open Software Suite (2000) Rice, P. Longden, I. and Bleasby, A. Trends in Genetics 16, (6) pp 276-277, http://emboss.bioinformatics.nl/). For protein sequences EBLOSUM62 is used for the substitution matrix. For nucleotide sequence, EDNAFULL is used. The optional parameters used are a gap-open penalty of 10 and a gap extension penalty of 0.5. The skilled person will appreciate that all these different parameters will yield slightly different results but that the overall percentage identity of two sequences is not significantly altered when using different algorithms.

After alignment by the program NEEDLE as described above the percentage of sequence identity between a query sequence and a sequence of the invention is calculated as follows: Number of corresponding positions in the alignment showing an identical amino acid or identical nucleotide in both sequences divided by the total length of the alignment after subtraction of the total number of gaps in the alignment. The identity defined as herein can be obtained from NEEDLE by using the NOBRIEF option and is labeled in the output of the program as “longest-identity”.

The nucleic acid and protein sequences of the present invention can further be used as a “query sequence” to perform a search against public databases to, for example, identify other family members or related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to nucleic acid molecules as described herein. BLAST protein searches can be performed with the XBLAST program, score=50, wordlength=3 to obtain amino acid sequences homologous to protein molecules as described herein. To obtain gapped alignments for comparison purposes, Gapped BLAST can be utilized as described in Altschul et al., (1997) Nucleic Acids Res. 25(17): 3389-3402. When utilizing BLAST and Gapped BLAST programs, the default parameters of the respective programs (e.g., XBLAST and NBLAST) can be used. See the homepage of the National Center for Biotechnology Information at http://www.ncbi.nlm.nih.gov/.

### Embodiments of the Invention

1. A recombinant host comprising a recombinant nucleic acid sequence encoding a polypeptide having:
   a. the amino acid sequence set forth in SEQ ID NO: 1 or an amino acid sequence having at least about 30% sequence identity thereto;
   b. the amino acid sequence set forth in SEQ ID NO: 2 or an amino acid sequence having at least about 30% sequence identity thereto;
   c. the amino acid sequence set forth in SEQ ID NO: 3 or an amino acid sequence having at least about 30% sequence identity thereto; or
   d. the amino acid sequence set forth in SEQ ID NO: 4 or an amino acid sequence having at least about 30% sequence identity thereto.
2. A recombinant host according to embodiment 1 which is capable of producing a glycosylated diterpene, such as a steviol glycoside.
3. A recombinant host according to embodiment 1 or 2 which comprises one or more recombinant nucleotide sequence(s) encoding:
   a polypeptide having ent-copalyl pyrophosphate synthase activity;
   a polypeptide having ent-Kaurene synthase activity;
   a polypeptide having ent-Kaurene oxidase activity; and
   a polypeptide having kaurenoic acid 13-hydroxylase activity.
4. A recombinant host according to any one of the preceding embodiments, which comprises a recombinant nucleic acid sequence encoding a polypeptide having NADPH-cytochrome p450 reductase activity.
5. A recombinant host according to any one of the preceding embodiments which comprises a recombinant nucleic acid sequence encoding one or more of:
   (i) a polypeptide having UGT74G1 activity (UGT3 activity);
   (ii) a polypeptide having UGT85C2 activity (UGT1 activity); and
   (iii) a polypeptide having UGT76G1 activity (UGT4 activity).
6. A recombinant host according to any one of the preceding embodiments which comprises a recombinant nucleic acid sequence encoding an additional polypeptide having UGT2 activity.
7. A recombinant host according to any one of the preceding embodiments, wherein the host belongs to one of the genera Saccharomyces, Aspergillus, Pichia, Kluyveromyces, Candida, Hansenula, Humicola, Issatchenkia, Trichosporon, Brettanomyces, Pachysolen, Yarrowia, Yamadazyma or Escherichia.
8. A recombinant host according to embodiment 7, wherein the recombinant host is a Saccharomyces cerevisiae cell, a Yarrowia lipolitica cell, a Candida krusei cell, an Issatchenkia orientalis or an Escherichia coli cell.
9. A recombinant host according to any one of the preceding embodiments, wherein the ability of the host to produce geranylgeranyl diphosphate (GGPP) is upregulated.
10. A recombinant host according to any one of the preceding embodiments, comprising one or more recombinant nucleic acid sequence(s) encoding hydroxymethylglutaryl-CoA reductase, farnesyl-pyrophosphate synthetase and geranylgeranyl diphosphate synthase.
11. A recombinant host according to any one of the preceding embodiments which comprises a nucleic acid sequence encoding one or more of:
   a polypeptide having hydroxymethylglutaryl-CoA reductase activity;
   a polypeptide having farnesyl-pyrophosphate synthetase activity;
   a polypeptide having geranylgeranyl diphosphate synthase activity.
12. A process for the preparation of a glycosylated diterpene which comprises fermenting a recombinant host according to any one of embodiments 2 to 11 in a suitable fermentation medium, and optionally recovering the glycosylated diterpene.
13. A process according to embodiment 12 for the preparation of a glycosylated diterpene, wherein the process is carried out on an industrial scale.
14. A fermentation broth comprising a glycosylated diterpene obtainable by the process according to embodiment 12 or 13.
15. A glycosylated diterpene obtained by a process according to embodiment 12 or 13 or obtainable from a fermentation broth according to embodiment 14.
16. A composition comprising two or more glycosylated diterpenes obtained by a process according to embodiment 12 or 13 or obtainable from a fermentation broth according to embodiment 14.
17. A foodstuff, feed or beverage which comprises a glycosylated diterpene according to embodiment 15 or a composition according to embodiment 16.
18. A method for converting a first glycosylated diterpene into a second glycosylated diterpene, which method comprises:
   contacting said first glycosylated diterpene with a recombinant host according to any one of embodiments 1 to 11, a cell free extract derived from such a recombinant host or an enzyme preparation derived from either thereof;
   thereby to convert the first glycosylated diterpene into the second glycosylated diterpene.
19. A method according to embodiment 18, wherein the second glycosylated diterpene is: stevio-19-diside, steviolbioside, stevioside, 13-[(β-D-Glucopyranosyl)oxy)kaur-16-en-18-oic acid 2-O-β-D-glucopyranosyl-β-D-glucopyranosyl ester, RebE or RebD.
20. A method according to embodiment 19, wherein the first glycosylated diterpene is stevio-13-monoside, steviol-19-monoside, rubusoside, stevioside, Rebaudioside A or 13-[(β-D-Glucopyranosyl)oxy)kaur-16-en-18-oic acid 2-O-β-D-glucopyranosyl-β-D-glucopyranosyl ester and the second glycosylated diterpene is steviol-19-diside, steviolbioside, stevioside, 13-[(β-D-Glucopyranosyl)oxy)kaur-16-en-18-oic acid 2-O-β-D-glucopyranosyl-β-D-glucopyranosyl ester, RebE or RebD.

A reference herein to a patent document or other matter which is given as prior art is not to be taken as an admission that that document or matter was known or that the information it contains was part of the common general knowledge as at the priority date of any of the claims.

The disclosure of each reference set forth herein is incorporated herein by reference in its entirety.

The present invention is further illustrated by the following Examples:

### EXAMPLES

### Example 1

### Construction of E. coli Expression Vectors

The full length open reading frame encoding UGTs from Solanum lycopersicon were amplified from S. lycopersicon cDNA. 1 μg of total RNA isolated from tomato fruit was used as starting material to prepare cDNA using the SMART™ RACE cDNA Amplification Kit (Clontech), according to the manufacturer's instructions.

For amplification Phusion “proofreading polymerase” (Finnzymes) and the primers mentioned in Table 1 were used.

TABLE 1
primers used to amplify tomato and stevia UGT fraqments

| | Forward primer | Reverse primer |
|---|---|---|
| RT7 | ATTAGGATCCAATGGGAACACAAGTAACAGAG | AATACTGCAGTTAATTAGTACTAATCTTACAAAATT |
| | | G |
| Rh11 | ATTAGGATCCAATGGAAGCCAAGAAAAATAAAATGAG | AATACTGCAGTCATTTGTTGCTGCAAAGAGCCATC |
| Rh15 | ATTAGGATCCAATGGATGGTTCGAATGAAAAGTC | AATACTGCAGCTAGACAACATTTGATCTAGTCTTG |
| Rh18 | ATTAGGATCCAATGAGTACTACTTTAAAGGTATTGAT | AATACTGCAGATTCACTTATTACTATTCCTACAAAG |
| | G | |
| UGT2_1a | ATTAGGATCCAATGGCCACTTCTGACTCCAT | AATAAAGCTTTTAGCTTTCGTGGTCAATGGCA |
| 85C2 | ATTAGGATCCAATGGACGCTATGGCCACCACT | AATAAAGCTTTTAGTTTCGAGCCAAGACAGTG |

The amplified fragment and vector pACYC-DUET1 (Novagen) were digested with the restriction enzymes BamHI and PstI for the tomato UGT fragments or BamHI and HindIII for UGT2_1a and UGT85C2, followed by purification of the required DNA fragments, their subsequent ligation and finally transformation into E. coli XL-1 blue using standard procedures. Recombinant bacteria were selected on LB plates containing 50 μg/mL chloramphenicol. After ON growth of recombinant colonies in liquid culture (3 mL LB broth with 50 μg/mL chloramphenicol, 250 rpm, 37° C.), plasmid DNA was isolated using the Qiaprep Spin Miniprep kit (Qiagen). Isolated plasmid material was checked by Sanger sequencing with vector primers.

This cloning strategy led to constructs from which the UGTs can be expressed with an N-terminal Hiss-tag

### Example 2

### Synthesis of Steviolmonoside by UGT85C2

To prepare Steviolmonoside enzymatically from Steviol (Sigma U4625) and UDP-glucose (Sigma 4625), the following compounds were mixed in a total reaction volume of 4 ml. For preparation of a crude enzyme extract of UGT85C2 see Example 3.

| | μl |
|---|---|
| 100 mM 2-mercaptoethanol in 0.1M Tris-buffer | 160 |
| 100 mM UDP-glucose in 10% DMSO | 800 |
| 100 mM Steviol in 100% DMSO | 40 |
| Crude enzyme extract UGT85C2 | 400 |

The glycosylation reaction was performed overnight at 30° C. and 100 rpm. Subsequently the reaction was purified using an Oasis hydrophiliclipophilic-Balanced (HLB) 3 cc extraction cartridge (Waters), which had been preconditioned according to the manufacturer's instructions. The enzymatic reaction was loaded on the HLB column, and allowed to enter the column by gravity flow. Subsequently the column was washed with 6 mL of water. Product was eluted by passing 3 ml of 100% methanol over the column. The methanol elute was dried under vacuum centrifugation and the pellet dissolved in 80 μl DMSO. This resulted in a 50 mM steviolmonoside preparation.

### Example 3

### In Vitro Comparison of Different Tomato UGTs and Stevia UGT2 1a

The control plasmid pACYC-DUET-1 and the UGT constructs were transformed to E. coli BL21 DE3 (Invitrogen). For expression, a 3 mL overnight culture of the recombinant E. coli strains was prepared (LB medium with appropriate antibiotic; 50 μg chloramphenicol/mL and 1% glucose). 200 μL of that culture was transferred to 20 mL of LB medium with the appropriate antibiotic in a 100 mL Erlenmeijer flask, and incubated at 37° C., 250 rpm until the A600 was 0.4 to 0.6. Subsequently IPTG was added to a final concentration of 1 mM and cultures were incubated overnight at 18° C. and 250 rpm. The next day, cells were harvested by centrifugation (10 min 8000×g), medium was removed, and cells were resuspended in 1 mL Resuspension buffer (100 mM Tris-HCl pH=7.5, 1.4 mM 2-mercaptoethanol; 4° C., 15% glycerol). Cells were disrupted by two times shaking with 200 mg 0.1 mm Zirconia/Silica Beads (BioSpec) for 10 seconds in a FastPrep FP120 machine (Savant) at speed 6.5. Insoluble particles were subsequently removed by centrifugation (10 min 13,000×g, 4° C.). The resulting supernatants were referred to as crude enzyme extracts.

### Example 4

### Glucosylation of Steviolmonoside and RebaudiosideA by UGTs

For enzyme assays, a mix of total 50 μl was made in a 2 ml eppendorf tube:

| | |
|---|---|
| 0.1M Tris in 2% DMSO | 37.5 μl |
| 100 mM 2-mercaptoethanol in 0.1M tris | 2 μl |
| 100 mM UDP-glucose in 10% DMSO | 5 μl |
| 50 mM Steviolmonoside in 100% DMSO | 0.5 μl |
| Crude UGT enzyme extract | 5 μl |

The tubes were incubated overnight at 30° C. and 100 rpm.

For assays with Rebaudiosise-A (RebA), Steviolmonoside was replaced by 0.5 μl 50 mM RebA (ChromDex ASB-00018225) in 50% DMSO.

### Example 5

### LC-MS Analyses

An LC-PDA-QTOF-MS system was used to analyse reaction products. After incubation, the in vitro enzyme assay mix (50 μl) was stopped by addition of 150 μl of 100% methanol in MQ water acidified with 0.13% formic acid. Samples were sonicated for 15 min, centrifuged at 2500 rpm for 10 min and filtered through 0.45 μm filters (Minisart SRP4, Biotech GmbH, Germany). For chromatographic separation, a Luna C18(2) pre-column (2.0×4 mm) and an analytical column (2.0×150 mm, 100 Å, particle size 3 μm) from Phenomenex (Torrance, Calif. USA) were used. Five microliters of each filtered sample were injected into the system for LC-PDA-MS analysis using formic acid : water (1:1000, v/v; eluent A) and formic acid : acetonitrile (1:1000, v/v; eluent B) as elution solvents. Flow was set at 0.19 mL/min with the gradient from 80% eluent A and 20% eluent B to 45% Eluent A and 55% eluent B across a period of 45 min. The column temperature was maintained at 40° C. and the samples at 20° C. UV absorbance was measured using a Waters 2996 PDA (λ range from 240 to 600 nm) and ESI-MS analysis was performed using a QTOF Ultima V4.00.00 mass spectrometer (Waters-Corporation, MS technologies) in negative mode. A collision energy of 10 eV was used for full-scan LC-MS in the m/z range 100 to 1,500. Leucine enkephalin ([M−H] ⁻=554.2620), was used for online mass calibration (lock mass).

Compounds were identified by their retention time and their apparent mass and compared to standard steviosides present in the Rebaudioside-A Impurities Mix-6 (Cerilliant S-017) (Table 2).

TABLE 2
Retention time and masses of steviosides in
the Rebaudioside-A Impurities Mix-6

| | Rt (min) | m/z |
|---|---|---|
| RebD | 12.95 | 1127.47 |
| RebA | 18.76 | 965.42 |
| Stevioside | 18.94 | 803.37 |
| Rubusoside | 22.84 | 803.37 |
| RebB | 25.15 | 641.31 |
| Steviolbioside | 25.58 | 641.31 |
| Steviol | 44.73 | 317.21 |

The results of the in vitro tests are given in Table 3 and 4. To semi-quantify the produced compounds of the in vitro assays, the peak surface area for each relevant peak was measured from the total ion count chromatograms . Clearly, UGT2_1a was able to produce steviolbioside (Rt=25.6) from Steviolmonoside. UGT RT18 also produces predominantly steviolbioside. Other RTs produce preferentially other steviolglycosides (Table 3).

TABLE 3
Products detected by LC-MS after in vitro reaction of Steviolmonoside
with different UGT enzymes. As substrate, Steviolmonoside
(Rt = 30.4 min; m/z 959 = [2M − H]) was
used. Shown are peak surface area in the LC-MS chromatograms.

| | m/z = | m/z = | m/z = | m/z = | m/z = | m/z = | m/z = |
|---|---|---|---|---|---|---|---|
| | 1011 | 407 | 803 | 803 | 641 | 641 | 641 |
| | Rt = | Rt = | Rt = | Rt = | Rt = | Rt = | Rt = |
| | 14.0 | 19.0 | 20.7 | 20.8 | 24.8 | 25.6 | 26.3 |
| blanc | | | | | | 2 | |
| UGT2_1a | | 23 | | | | 11532 | |
| RT18 | | | | | 153 | 4408 | |
| RT15 | | | 540 | | 5841 | 8467 | 1 |
| RT11 | 3 | | | 10110 | 16229 | 2163 | |
| RT7 | | | | 163 | 7764 | 1788 | |

Rt: retention time in minutes. Steviolbioside is detected at 25.6 min.

When testing RebA as a substrate, it was clear that RT18 showed a relatively strong formation of RebD (Rt=12.9 min) from RebA, in comparison with UGT2_1a, while the other UGTs preferentially produce different RebA-glycosides (Table 4).

TABLE 4
Products detected by LC-MS after in vitro reaction of RebA
with different UGT enzymes. As substrate, RebA (Rt =
18.74 min; m/z 1011 = [M − H + formic acid])
was used. Shown are peak surface area in the LC-MS chromatograms.

| | m/z = | m/z = | m/z = | m/z = | m/z = | m/z = |
|---|---|---|---|---|---|---|
| | 565 | 1127 | 1127 | 1127 | 1127 | 1127 |
| | Rt = | Rt = | Rt = | Rt = | Rt = | Rt = |
| | 12.6 | 12.9 | 14.1 | 14.25 | 14.9 | 17.4 |
| Blanc | | 6 | | 12 | | |
| UGT2_1a | | 1037 | 16 | | | |
| RT18 | 98 | 3596 | | 494 | | |
| RT15 | | 376 | | 8946 | 234 | |
| RT11 | | 33 | | 7684 | 159 | |
| RT7 | | 1212 | | 2169 | | 31 |

Rt: retention time in minutes. RebD is detected at Rt = 12.9 min.

Thus, RT18 can form steviolbioside from steviolmonoside, and RebD from RebA.

### Example 6

### UGT Protein Content in Crude-Enzyme Extracts

We observed that the activity for the formation of steviolbioside of the RT18 crude enzyme extract was 2-3 fold lower compared to UGT2_1a. To be able to compare the two enzymes for the activity per enzyme molecule in the crude extracts, we analysed the total protein content of the crude enzyme extracts.

First, the extracts were compared for protein content using Protein Dye Reagent concentrate (BIO-RAD 500-0006), according to the manufacturer's instructions, using lyophilized Bovine Serum Albumine BioRad 500-0007) as a standard. Based on this it was observed that UGT2_1a crude extract contained twice as much protein as the RT18 crude extract (Table 5).

TABLE 5
Total protein content of crude enzyme extracts.
Protein concentration is given in μg/μl

| | Total protein (μg/μl) |
|---|---|
| pACYC-DUET-1 | 3.35 |
| UGT2_1a | 5.08 |
| RT18 | 2.66 |
| RT15 | 3.63 |
| RT11 | 3.33 |
| RT7 | 2.06 |

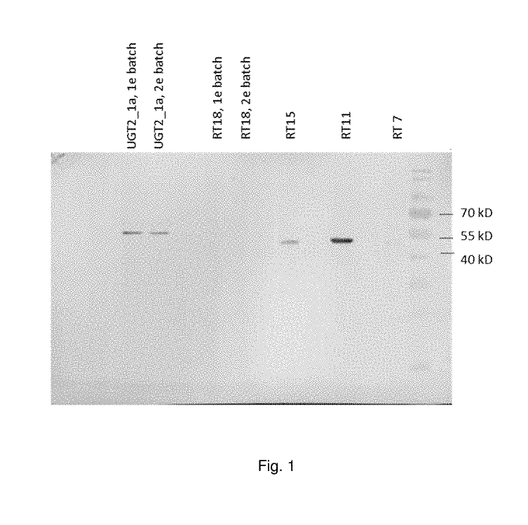

Subsequently, to compare the enzyme concentrations in the crude extracts, a western blot experiment was performed. 50 μg of total protein was brought in 50 μl Sample buffer (20 mM Tris pH 6.8, 6% glycerol, 0.4% SDS, 20 mM Dithiothreitol, 0.01% Bromophenol Blue) and boiled for 5 minutes. Subsequently 10 μl sample (=10 μg total protein) was loaded on a 12.5% poly-acryl amide gel with SDS and run for 2 hours at 20 mA. Proteins were transferred from the gel onto nitrocellulose membrane (BIO-RAD) in standard blotting buffer (3 g/L Tris, 14.4 g/L glycine, 10% ethanol) for 1 hour (100 V). The nitrocellulose was subsequently washed with TBST buffer (20 mM Tris-Cl buffer pH 7.5, 150 mM NaCl, 0.05% Tween 20) for 5 minutes, and blocked with TBST buffer with 2% non-fat dry milk powder (ELK) for 1 hour. The presence of enzyme was detected by incubation for 1 hour with TBST with 2% ELK and 1:4000 diluted antiHis monoclonal antibody conjugated to peroxidase (Sigma, St Louis, A7058). After washing four times five minutes with TBST, the peroxidase was detected by the TMB Liquid substrate system for membranes (Sigma T0565). A purple colour was detected at the position where His-tagged proteins (here: UGTs) were present on the blot. When all five crude enzyme extracts were compared in this way (FIG. 1) it was clear that UGT2_1a was expressed to well-detectable levels, while RT18 protein could not be detected. The other UGTs (RT15, RT11,RT7) were also detected, to different intensities.

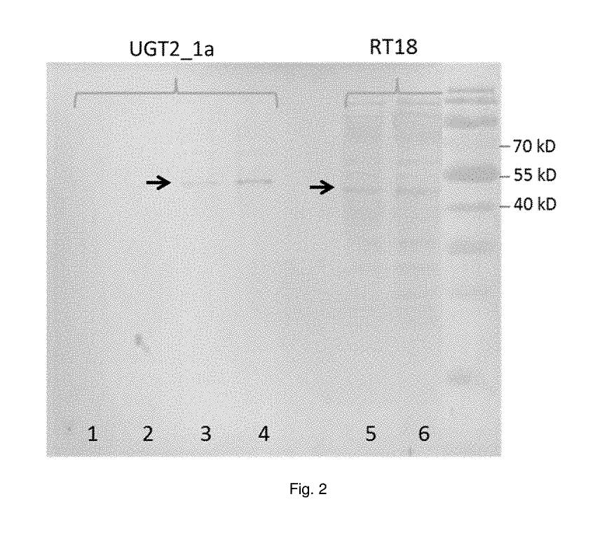

To compare the UGT content in the crude enzyme extracts of RT18 and UGT2_1a, another western blot was made. For UGT2_1a, 0.5, 1.0, 1.9, 3.8 μg protein was loaded, while for RT18, 31.9 and 63.8 μg was loaded. Detection of UGTs was performed as described above. The blot (FIG. 2) showed that the amount of His-tagged UGT protein was the same in 1.9 μg UGT2_1a extract and 63.8 μg RT18, as estimated by visual inspection. This indicated that the concentration of UGT protein in the RT18 crude extract was 20-50 fold lower than in the UGT2_1a crude extract. Thus, the activity of RT18, as recorded in Tables 3 and 4, is more than 10-fold higher than UGT2_1a when using steviolmonoside as a substrate, and more than 50-fold higher when using RebA as a substrate.

### Example 7

### MSMS Analysis

To provide more evidence that the products of UGT2_1a and RT18 with steviolmonoside as a substrate were identical, the steviolbioside product of RT18 was further compared to the steviolbioside product from UGT2_1a and the steviol-diglucoside product from RT11 by tandem mass spectrometry analysis (LC-MS²). The methanol extracts from the RT18 and UGT2_1a enzyme assays were injected in an Accela HPLC-PDA (Thermo) coupled to a LTQ Ion Trap-Orbitrap FTMS hybrid mass spectrometer (Thermo) system was used. Data-directed MSMS was performed using the same LC conditions as described for LC-QTOF MS analysis (see above), and using negative ionization mode, with an Isolation Width of 3.00 Dalton and a Normalized Collission Energy of 35.0. Retention times of steviolglucosides differed slightly from the analysis on the LC-QTOF MS system (see above, Table 3).

Fragmentation was performed on the compounds with m/z 641.30 eluting at 23.0 min in the RT18 and UGT2_1a samples and eluting at 22.2 min in the RT11 sample.

In the fragmentation spectra of ions of m/z 641.30, the fragments m/z 479.26 [M−H−Glucose] and m/z 317.21 [M−H−2Glucose] were observed in all three samples. The ratio between the m/z 317.21 and m/z 479.26 ions was recorded for all three compounds. For both the RT18 and UGT2_1a compounds, the ratio m/z 317 to m/z 479 was 2:10, while the the ratio m/z 317 to m/z 479 for the RT11 compound was 4:10. Thus, the MS2 analysis did not differentiate the steviolbioside products from RT18 and UGT2_1a, but did differentiate the RT11 steviol diglucoside product from these two. These results further confirm that the major product of RT18 corresponds to steviolbioside.

### Example 8

### RT18 Expression in Steviol Glycoside Production Strain

In order to demonstrate the effect of the in vivo activity of the RT18 enzyme on the production of steviol glycosides, RT18 (SEQ ID NO: 19) was assembled with three promoters of different strength (Table 6), and transformed to a Yarrowia lipolitica strain that produces steviol glycosides using the approach described in WO2013/110673 and WO2015/007748. The genotype of this Yarrowia strain is given in Table 7

TABLE 6
Different strength promotors used for RT18 expression

| Relative promoters strength | Name |
|---|---|
| Weak | CWP (SEQ ID NO: 20) |
| Medium | SCP2 (SEQ ID NO: 21) |
| Strong | HSP (SEQ ID NO: 22) |

TABLE 7
Genotype of parental strain (copy number; SEQ ID NO).

| | |
|---|---|
| Parent | MATB tHMG (2; SEQ ID NO: 23) GGS (3; SEQ ID NO: 24) |
| strain | CPS (5; SEQ ID NO: 25) KS (4; SEQ ID NO: 26) KO (3; |
| genotype | SEQ ID NO: 27) KAH4 (4; SEQ ID NO: 28) CPR (2; SEQ ID |
| | NO: 29) UGT1 (3; SEQ ID NO: 30) UGT2 (2; SEQ ID NO: |
| | 31) UGT3 (2; SEQ ID NO: 32) UGT4 (3; SEQ ID NO: 33) |

For positive transformants, a pre-culture was inoculated with colony material from YEPh-D agar. The pre-culture was grown in 200 μl YEP with glucose as carbon source. The pre-culture was incubated 72 hours in an Infors incubator at 30° C., 750 rpm and 80% humidity. 40 μl of pre-culture was used to inoculate 2.5 ml main culture. The main cultures were incubated 120 hours in an Infors incubator at 30° C., 550 rpm, 80% humidity. After 120 h the main culture was spun down at 2750 rpm for 10 min. Supernatant was diluted with water and acetonitrile, and measured using LC/MS.

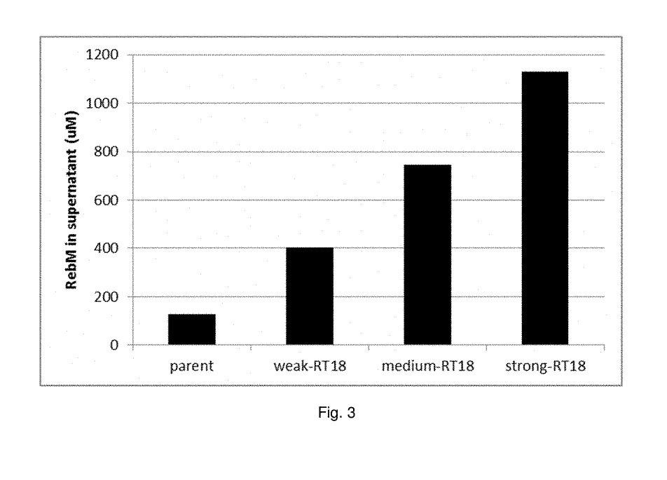

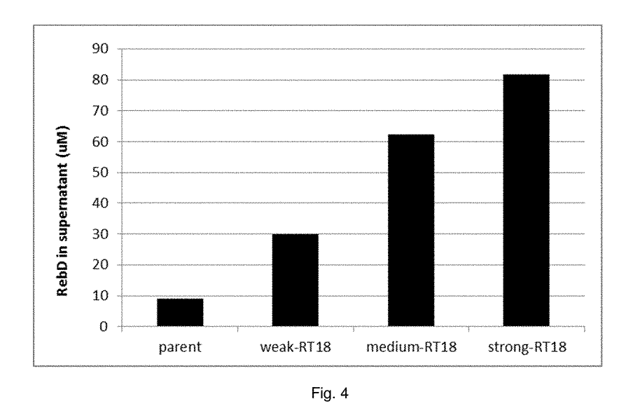

The results are set out in in FIGS. 3 and 4. It can be seen that the strains that express the RT18 produce higher amounts of RebM and RebD compared to the parent. In addition, the stronger the expression, the more RebD and RebM were produced. The formation of higher RebD illustrates RT18 is effective in catalyzing the glycosylation of the glucose on the 19-position of steviol glycosides (see FIG. 6), for example catalyzing the formation of RebD from RebA. RebD can then be further converted to RebM, catalyzed by UGT4.

### Example 9

### RT18 and UGT4 Expression in Steviol Glycoside Production Strain

The expression of other UDP-glycosyl transferases, in combination with RT18, will have an influence on the product profile. For example the RebD that is over-produced in a strain expressing RT18 can be further converted to RebM by the activity of UGT4. In order to evaluate the effect of over-expression of RT18 with UGT4, expression vectors of RT18 and UGT4 were transformed to a Yarrowia lipoitica strain producing steviol glycosides using the approach described in WO2013/110673 and WO2015/007748. The genotype of this parental strain is given in Table 8.

TABLE 8
Genotype of strain used to transform RT18
and UGT4 (copy number; SEQ ID NO)

| | |
|---|---|
| Parent | MATB tHMG (2; SEQ ID NO: 23) GGS (2; SEQ ID NO: 24) |
| strain | CPS (2; SEQ ID NO: 25) KS (2; SEQ ID NO: 26) KO (2; |
| genotype | SEQ ID NO: 27) KAH4 (2; SEQ ID NO: 28) CPR (2; SEQ |
| | ID NO: 29) UGT1 (2; SEQ ID NO: 30) UGT2 (1; SEQ ID |
| | NO: 34) UGT3 (2; SEQ ID NO: 32) UGT4 (2; |
| | SEQ ID NO: 33) |

For positive transformants, a pre-culture was inoculated with colony material from YEPh-D agar. The pre-culture was grown in 200 μl YEP with glucose as carbon source. The pre-culture was incubated 72 hours in an Infors incubator at 30° C., 750 rpm and 80% humidity. 40 μl of pre-culture was used to inoculate 2.5 ml main culture. The main cultures were incubated 120 hours in an Infors incubator at 30° C., 550 rpm, 80% humidity. After 120 h the main culture was spun down at 2750 rpm for 10 min. Supernatant was diluted with water and acetonitrile, and measured using LC/MS.

The results are set out in Table 9, where the percentages of steviol glycosides on total steviol glycosides are listed for the two strains. It can be seen that the strains that expresses the RT18 in combination with additional UGT4 effectively convert a higher percentage of the steviol glycosides to higher glycosylated steviol glycosides. Particularly, RebB, Stevioside and RebA are lower in the strain expressing the RT18 and UGT4, whereas the abundance of RebM is greatly increased. This illustrates the effectiveness of RT18 expression in steering steviol glycoside production towards higher glycosylated products such as RebM.

TABLE 9
Percentages of steviol glycosides of total steviol glycosides in parent
strain and strain expressing RT18 and an extra copy of UGT4.

| | | | | | | Other |
|---|---|---|---|---|---|---|
| | | | | | | steviol |
| Strain | RebM | RebD | RebA | Stevioside | RebB | glycosies |
| parent | 3 | 6 | 54 | 25 | 7 | 6 |
| RT18, UGT4 | 66 | 5 | 20 | 1 | 1 | 6 |

TABLE 10
Description of the sequence listing

| SEQ ID NO | Description |
|---|---|
| SEQ ID NO: 1 | amino acid sequence of the RT7 protein from Solanum lycopersicon |
| | (Solyc11g007480 - tomato genome: http://solgenomics.net/) |
| SEQ ID NO: 2 | amino acid sequence of the RT11 protein from Solanum lycopersicon |
| | (Solyc11g007500) |
| SEQ ID NO: 3 | amino acid sequence of the RT15 protein from Solanum lycopersicon |
| | (Solyc04g081830) |
| SEQ ID NO: 4 | amino acid sequence of the RT18 protein from Solanum lycopersicon |
| | (Solyc05g005930) |
| SEQ ID NO: 5 | amino acid sequence of the UGT85C2 protein from Stevia rebaudiana |
| SEQ ID NO: 6 | amino acid sequence of the UGT2_1a protein from Stevia rebaudiana |
| SEQ ID NO: 7 | nucleic acid sequence of the RT7 forward primer |
| SEQ ID NO: 8 | nucleic acid sequence of the RT7 reverse primer |
| SEQ ID NO: 9 | nucleic acid sequence of the RT11 forward primer |
| SEQ ID NO: 10 | nucleic acid sequence of the RT11 reverse primer |
| SEQ ID NO: 11 | nucleic acid sequence of the RT15 forward primer |
| SEQ ID NO: 12 | nucleic acid sequence of the RT15 reverse primer |
| SEQ ID NO: 13 | nucleic acid sequence of the RT18 forward primer |
| SEQ ID NO: 14 | nucleic acid sequence of the RT18 reverse primer |
| SEQ ID NO: 15 | nucleic acid sequence of the UGT2_1a forward primer |
| SEQ ID NO: 16 | nucleic acid sequence of the UGT2_1a reverse primer |
| SEQ ID NO: 17 | nucleic acid sequence of the UGT85C2 forward primer |
| SEQ ID NO: 18 | nucleic acid sequence of the UGT82C2 reverse primer |
| SEQ ID NO: 19 | nucleic acid sequence of the RT18 open reading frame optimized for |
| | expression in Y. lipolitica |
| SEQ ID NO: 20 | nucleic acid sequence of CWP promoter from Y. lipolitica |
| SEQ ID NO: 21 | nucleic acid sequence of SCP2 promoter from Y. lipolitica |
| SEQ ID NO: 22 | nucleic acid sequence of HSP promoter from Y. lipolitica |
| SEQ ID NO: 23 | nucleic acid sequence of tHMG optimized for expression in Y. lipolitica |
| SEQ ID NO: 24 | nucleic acid sequence of GGS optimized for expression in Y. lipolitica |
| SEQ ID NO: 25 | nucleic sequence of CPS from S. rebaudiana optimized for expression in Y. lipolitica |
| SEQ ID NO: 26 | nucleic acid sequence of tKS from S. rebaudiana optimized for expression in |
| | Y. lipolitica |
| SEQ ID NO: 27 | nucleic acid sequence of KO from Gibberella fujikori optimized for expression |
| | in Y. lipolitica |
| SEQ ID NO: 28 | nucleic acid sequence of KAH_4 optimized for expression in Y. lipolitica |
| SEQ ID NO: 29 | nucleic acid sequence of CPR_optimized for expression in Y. lipolitica |
| SEQ ID NO: 30 | nucleic acid sequence of UGT1 optimized for expression in Y. lipolitica |
| SEQ ID NO: 31 | nucleic acid sequence of UGT2 variant optimized for expression in Y. lipolitica |
| SEQ ID NO: 32 | nucleic acid sequence of UGT3 optimized for expression in Y. lipolitica |
| SEQ ID NO: 33 | nucleic acid sequence of UGT4 optimized for expression in Y. lipolitica |
| SEQ ID NO: 34 | nucleic acid sequence of UGT2 variant optimized for expression in Y. lipolitica |

1. A recombinant host comprising a recombinant nucleic acid sequence encoding a polypeptide comprising:
a. the amino acid sequence set forth in SEQ ID NO: 1 or an amino acid sequence having at least about 30% sequence identity thereto;
b. the amino acid sequence set forth in SEQ ID NO: 2 or an amino acid sequence having at least about 30% sequence identity thereto;
c. the amino acid sequence set forth in SEQ ID NO: 3 or an amino acid sequence having at least about 30% sequence identity thereto; or
d. the amino acid sequence set forth in SEQ ID NO: 4 or an amino acid sequence having at least about 30% sequence identity thereto.

2. A recombinant host according to claim 1 which is capable of producing a glycosylated diterpene.

3. A recombinant host according to claim 1 which comprises one or more recombinant nucleotide sequence(s) encoding:
a polypeptide having ent-copalyl pyrophosphate synthase activity;
a polypeptide having ent-Kaurene synthase activity;
a polypeptide having ent-Kaurene oxidase activity; and/or
a polypeptide having kaurenoic acid 13-hydroxylase activity.

4. A recombinant host according to claim 1, which comprises a recombinant nucleic acid sequence encoding a polypeptide having NADPH-cytochrome p450 reductase activity.

5. A recombinant host according to claim 1 which comprises a recombinant nucleic acid sequence encoding one or more of:
(i) a polypeptide having UGT74G1 activity (UGT3 activity);
(ii) a polypeptide having UGT85C2 activity (UGT1 activity); and/or
(iii) a polypeptide having UGT76G1 activity (UGT4 activity).

6. A recombinant host according to claim 1 which comprises a recombinant nucleic acid sequence encoding an additional polypeptide having UGT2 activity.

7. A recombinant host according to claim 1, wherein the host belongs to one of the genera Saccharomyces, Aspergillus, Pichia, Kluyveromyces, Candida, Hansenula, Humicola, Issatchenkia, Trichosporon, Brettanomyces, Pachysolen, Yarrowia, Yamadazyma or Escherichia.

8. A recombinant host according to claim 7, wherein the recombinant host is a Saccharomyces cerevisiae cell, a Yarrowia lipolitica cell, a Candida krusei cell, an Issatchenkia orientalis or an Escherichia coli cell.

9. A recombinant host according to claim 1, wherein the ability of the host to produce geranylgeranyl diphosphate (GGPP) is upregulated.

10. A recombinant host according to claim 1, comprising one or more recombinant nucleic acid sequence(s) encoding hydroxymethylglutaryl-CoA reductase, farnesyl-pyrophosphate synthetase and/or geranylgeranyl diphosphate synthase.

11. A recombinant host according to claim 1 which comprises a nucleic acid sequence encoding one or more of:
a polypeptide having hydroxymethylglutaryl-CoA reductase activity;
a polypeptide having farnesyl-pyrophosphate synthetase activity; and/or
a polypeptide having geranylgeranyl diphosphate synthase activity.

12. A process for preparation of a glycosylated diterpene which comprises fermenting a recombinant host according to claim 2 in a suitable fermentation medium, and optionally recovering the glycosylated diterpene.

13. A process according to claim 12 for preparation of a glycosylated diterpene, wherein the process is carried out on an industrial scale.

14. A fermentation broth comprising a glycosylated diterpene obtainable by the process according to claim 12.

15. A glycosylated diterpene obtained by a process according to claim 12 or obtainable from a fermentation broth produced therefrom.

16. A composition comprising two or more glycosylated diterpenes obtained by a process according to claim 12 or obtainable from a fermentation broth produced therefrom.

17. A foodstuff, feed or beverage which comprises a glycosylated diterpene according to claim 15 or a composition thereof.

18. A method for converting a first glycosylated diterpene into a second glycosylated diterpene, which method comprises:
contacting said first glycosylated diterpene with a recombinant host according to claim 1, a cell free extract derived from such a recombinant host or an enzyme preparation derived from either thereof;
thereby to convert the first glycosylated diterpene into the second glycosylated diterpene.

19. A method according to claim 18, wherein the second glycosylated diterpene is: steviol-19-diside, steviolbioside, stevioside, 13-[(β-D-Glucopyranosyl)oxy)kaur-16-en-18-oic acid 2-O-β-D-glucopyranosyl-β-D-glucopyranosyl ester, RebE or RebD.

20. A method according to claim 19, wherein the first glycosylated diterpene is steviol-13-monoside, steviol-19-monoside, rubusoside, stevioside, Rebaudioside A or 13-[(β-D-Glucopyranosyl)oxy)kaur-16-en-18-oic acid 2-O-β-D-glucopyranosyl-μ-D-glucopyranosyl ester and the second glycosylated diterpene is stevio-19-diside, steviolbioside, stevioside, 13-[(βD-Glucopyranosyl)oxy)kaur-16-en-18-oic acid 2-O-β-D-glucopyranosyl-β-D-glucopyranosyl ester, RebE or RebD.
